(19) United States
(12) Patent Application Publication
(10) Pub. No.: US 2005/0260162 A1
(43) Pub. Date: Nov. 24, 2005

(54) Conditionally replicative adenovirus to target the Rb and Rb-related pathways

(76) Inventors: Juan Fueyo, Houston, TX (US); Athanassios P. Kyritsis, Stavraki (GR); Candelaria Gomez-Manzano, Houston, TX (US); W. K. Alfred Yung, Houston, TX (US); Polly S. Y. Lee, Houston, TX (US); Victor A. Levin, Houston, TX (US); Charles A. Conrad, Spring, TX (US); Frederick F. Lang JR., Houstoon, TX (US)

Correspondence Address: FULBRIGHT & JAWORSKI, LLP, 1301 MCKINNEY, SUITE 5100, HOUSTON, TX, 77010-3095, US

(21) Appl. No.: 11/080,248

(22) Filed: Mar. 15, 2005

Related U.S. Application Data
(62) Division of application No. 10/124,608, filed on Apr. 17, 2002, now abandoned.
(60) Provisional application No. 60/284,402, filed on Apr. 17, 2001.

(51) Int. Cl. C12Q 1/68; A61K 48/00; C12N 15/861

(52) U.S. Cl. 424/93.2; 435/456

(57) ABSTRACT

The present invention involves a method of treating cancer using a mutant adenovirus that is targeted to cells with a mutant retinoblastoma pathway. The mutant adenovirus is able to kill the tumor cells without harming the cells with a wild type retinoblastoma pathway.

This application is a divisional of co-pending application of Ser. No. 10/124,608, filed Apr. 17, 2002, which claims priority to U.S. Provisional Application Ser. No. 60/284,402, filed Apr. 17, 2001, the entire disclosures of which are specifically incorporated herein by reference.

### BACKGROUND OF THE INVENTION

1. Field of the Invention

The invention generally relates to the field of oncology and tumor-selective adenoviruses. More particularly, it concerns compositions and methods of treating cancer in a patient using oncolytic adenoviruses. The invention also concerns methods of screening for retinoblastoma pathway function, particularly in cancer patients.

2. Description of Related Art

The development of cancer is understood as the culmination of complex, multistep biological processes, occurring through the accumulation of genetic alterations. Many if not all of these alterations involve specific cellular growth-controlling genes. These genes typically fall into two categories: proto-oncogenes and tumor suppressor genes. Mutations in genes of both classes generally confer a growth advantage on the cell containing the altered genetic material.

The function of tumor suppressor genes, as opposed to proto-oncogenes, is to antagonize cellular proliferation. When a tumor suppressor gene is inactivated, for example by point mutation or deletion, the cell's regulatory machinery for controlling growth is upset. The studies of several laboratories have shown that the neoplastic tendencies of such mutated cells can be suppressed by the addition of a wild-type tumor suppressor gene (a gene without mutation) that expresses its gene product (Levine, 1995).

Mutations and/or loss of function in the retinoblastoma tumor suppressor gene have been associated with tumor formation. The various types of tumors include gliomas, sarcomas, brain, lung, breast, ovary, cervix, pancreas, stomach, colon, skin, larynx, bladder and prostate. In some instances brain tumors are metastases to the brain from a primary tumor outside of the central nervous system (CNS). Brain tumors derived from metastases are typically more common than primary tumors of the brain. The most common primary tumors that metastasize to the brain are lung, breast, melanoma, and kidney. These brain metastases are usually in multiple sites, but solitary metastases may also occur.

The term “glioma” refers to a tumor originating in the neuroglia of the brain or spinal cord. Gliomas are derived form the glial cell types such as astrocytes and oligodendrocytes, thus gliomas include astrocytomas and oligodendrogliomas, as well as anaplastic gliomas, glioblastomas, and ependymonas. Astrocytomas and ependymomas can occur in all areas of the brain and spinal cord in both children and adults. Oligodendrogliomas typically occur in the cerebral hemispheres of adults. Gliomas account for 75% of brain tumors in pediatrics and 45% of brain tumors in adults. The remaining percentages of brain tumors are meningiomas, ependymomas, pineal region tumors, choroid plexus tumors, neuroepithelial tumors, embryonal tumors, peripheral neuroblastic tumors, tumors of cranial nerves, tumors of the hemopoietic system, germ cell tumors, and tumors of the sellar region.

Gene therapy is a promising treatment for gliomas, as well as other brain tumors, because of the failure and toxicity of conventional therapies. In addition, the identification of genetic abnormalities contributing to malignancies is providing crucial molecular genetic information to aid in the design of gene therapies. Genetic abnormalities indicated in the progression of tumors include the inactivation of tumor suppressor genes and the overexpression of numerous growth factors and oncogenes. Tumors treatment may be accomplished by supplying a gene or other therapeutic that target the mutations and resultant aberrant physiologies of tumors. It is these mutations and aberrant physiology that distinguishes tumor cells from normal cells. A tumor-selective virus would be a promising tool for gene therapy. Recent advances in the knowledge of how viruses replicate have been used to design tumor-selective oncolytic viruses. In gliomas, three kinds of viruses have been shown to be useful in animal models: reoviruses that can replicate selectively in tumors with an activated ras pathway (Coffey et al., 1998); genetically altered herpes simplex viruses (Martuza et al., 1991; Mineta et al., 1995; Andreanski et al., 1997), including those that can be activated by the different expression of proteins in normal and cancer cells (Chase et al., 1998); and mutant adenoviruses that are unable to express the E1B55 kDa protein and are used to treat p53-mutant tumors (Bischof et al., 1996; Heise et al., 1997; Freytag et al., 1998; Kim et al., 1998). Taken together, these reports confirm the relevance of oncolytic viruses as anti-cancer agents. In all three systems, the goal is the intratumoral spread of the virus and the ability to selectively kill cancer cells. Genetically modified adenoviruses that target cellular pathways at key points have both potent and selective anti-cancer effects in gliomas.

Adenoviruses and the Rb and p53 pathways interact as follows. Adenoviruses synthesize proteins that compel infected cells to replicate the viral DNA and suppress the apoptosis inducing mechanism in infected cells. Typically, the E1A proteins are the first virus-specific polypeptides synthesized after adenoviral infection and are required for viral replication to occur (Dyson and Harlow, 1992; Flint and Shenk, 1997). The targets of E1A proteins, such as retinoblastoma protein (Rb), seem to modulate the cell cycle by regulating cell progression from G₀ and G₁ into S phase. The retinoblastoma protein acts as a tumor suppressor by binding to gene regulatory proteins that increase DNA replication. In the case of retinoblastoma (Rb), binding between the Rb protein and the E1A protein results in release of E2F from preexisting cellular E2F-Rb complexes. The E2F is then free to activate both the E2 promoter of the adenovirus and several cell cycle-regulatory genes. The transcriptional activation of these cellular genes in turn helps to create an environment suitable for viral DNA synthesis in otherwise quiescent cells (Nevins, 1992). Two segments of E1A are important for binding Rb; one includes amino acids 30-60 (CR1) and the other includes amino acids 120-127 (CR2) (Whyte et al., 1988; Whyte et al., 1989). Deletion of either region prevented the formation of detectable E1A/Rb complexes in vitro and in vivo (Whyte et al., 1989).

Targeting the Rb pathway has noted relevance for the treatment of gliomas because abnormalities of the p16/Rb/E2F pathway are present in most gliomas (Fueyo et al., 1998a; Gomez-Manzano et al., 1998). Targeting this pathway by replacement of lost tumor suppressor activity through the transfer of p16 and Rb genes has produced cytostatic effects (Fueyo et al., 1998a; Gomez-Manzano et al., 1998). Transfer of E2F-1 resulted in powerful anti-cancer effect since the exogenous wild-type E2F-1 induced apoptosis and inhibited tumor growth in vivo (Fueyo et al., 1998b). However, treating human glioma tumors with existing adenovirus constructs realistically cannot affect significant portions of the tumor, mainly because replication-deficient adenoviral vectors are unable to replicate and infect other cells, thus transferring the exogenous gene to sufficient numbers of cancer cells (Puumalainen et al., 1998). Although targeting the p16/Rb/E2F pathway produces an anti-cancer effect in vitro, this imperfection of the vector system limits the therapeutic effect of the gene in vivo.

There is a continued need for improvement in the treatment of cancer, particularly brain tumors, including improvements related to the creation of oncolytic viruses that are capable of cell-specific infection and that are effective in infection-resistant cells within the tumor.

### SUMMARY OF THE INVENTION

Therefore, the present invention provides an oncolytic adenovirus capable of killing target cells, such as a tumor cells, but not the surrounding non-tumor cells. The invention takes advantage of the discovery that an adenovirus encoding an E1A polypeptide unable to bind the tumor suppressor protein Rb may not replicate in or kill a cell that has a functional Rb pathway, but may replicate in and kill a cell that has a defective Rb pathway. The adenovirus can be delivered intracranially (into the skull cavity) or intravenously to a subject and exhibit a therapeutic effect on a tumor in the subject. The tumor may be a primary tumor or it may be a tumor resulting from a metastasis to the skull or brain.

The present invention provides, in some embodiments, methods of treating cancer in a patient comprising administering intracranially to a cell or cells in a patient an effective amount of a composition comprising a replication-competent adenovirus comprising a mutation in a nucleic acid sequence encoding an E1A, E1B, or an E1A and E1B polypeptide, wherein the E1A polypeptide is unable to bind Rb and the E1B polypeptide is unable to bind p53. In other embodiments, the composition may be administered to the patient intravenously. It is contemplated that a patient includes any animal, including mammals such as mice, rats, monkeys, and humans. As used herein, the term “cancer” refers to a disease or set of diseases characterized by hyperproliferative cells, that is, cells whose growth is no longer controlled compared to normal cells. The term “therapeutically effective” refers to an ability to effect a therapeutic (positive with respect to health) result. “Effective amount” refers to an amount that can effect a particular result; in the context of cancer. An effective amount of a pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease. Thus, in several embodiments of the invention, these terms are appropriate with respect to methods of treating cancer or any other detection method of the present invention.

Cancer cells are targets for the present invention, including tumor cells. The term glioma refers to brain tumors including: anaplastic gliomas, glioblastomas, astrocytomas and oligodendrogliomas, all of which are included as targets of the claimed invention. Gliomas are tumors that arise from glial cells. Types of glial cells include, but are not limited to astrocytes and oligodendrocytes.

In other embodiments, it is further contemplated that compositions are employed in methods of the present invention to confer a therapeutic benefit on a subject with cancer. The term “therapeutic benefit” used throughout this application refers to anything that promotes or enhances the well-being of the patient with respect to the medical treatment of his hyperproliferative disease. A list of nonexhaustive examples of this includes extension of the patient's life by any period of time; decrease or delay in the neoplastic development of the disease; decrease in hyperproliferation; reduction in tumor growth; delay of metastases; reduction in the proliferation rate of a cancer cell, tumor cell, or any other hyperproliferative cell; induction of apoptosis in any treated cell or in any cell affected by a treated cell; and a decrease in pain to the patient that can be attributed to the patient's condition.

In many embodiments of the invention, the methods are used in patients whose cancer is characterized by one or more cells having a mutated polypeptide in the Rb or p53 pathway. The terms “Rb pathway” and “p53 pathway” refer to sequences of reactions specifically involving the Rb polypeptide or the p53 polypeptide, respectively, to regulate growth in a cell. A polypeptide or protein in the pathways (“Rb pathway polypeptide” or “p53 pathway polypeptide”) are ones that plays a role in mediating a reaction that directly involves Rb or p53, respectively, either upstream or downstream from the reaction. The Rb polypeptide is a Rb pathway polypeptide. Thus, it is contemplated that methods and compositions of the claimed invention may be practiced with respect to the Rb, p53 or both Rb and p53 pathways, which includes other polypeptides of the respective pathways. The selective nature of the adenovirus of the invention is taken advantage of when the Rb, p53, or both the Rb and p53 pathways are mutated such that the E2 promoter is not repressed and/or the apoptotic pathways are suppressed.

The present invention concerns cells having a mutation that affects the Rb, the p53, or the Rb and the p53 pathways. Thus, it is contemplated that cells may harbor a mutation that alters the expression, degradation, stability, specificity, activity, function, location, folding/structure of Rb or an Rb pathway polypeptide and/or p53 or a p53 pathway polypeptide.

Methods of the present invention may further include steps for determining whether the cell or cells has a mutation in an Rb or p53 pathway polypeptide or in a gene encoding a polypeptide in the Rb or p53 pathway; in some embodiments a mutation in Rb or an Rb polypeptide is evaluated. It is contemplated that any assay may be employed that allows a mutation in a gene or protein, whether by evaluating genomic DNA, transcribed RNA, or translated protein, to be identified and/or characterized. In some embodiments, the activity of a Rb pathway polypeptide is assayed. The ability of Rb to inhibit E2F activation of transcription may be evaluated. Thus, an expression assay using a reporter gene under the control of an E2F element may be involved. It is contemplated that in some cases an antibody directed against the Rb pathway polypeptide is employed.

The present invention also concerns an adenovirus that has a mutation in the gene encoding E1A, E1B, or both E1A and E1B. One such mutation may be in a regulatory region or in a coding region for E1A (SEQ ID NO: 1). Other mutations may be in a regulatory or in a coding region for E1B (SEQ ID NO:3). While the adenovirus may carry a mutation anywhere in the E1A gene, it is specifically contemplated that mutations in the E1A gene that affect the ability of the E1A polypeptide to specifically bind the Rb polypeptide are part of the present invention. Thus, mutations contemplated include, but are not limited to, a mutation in the region encoding CR1 and/or CR2 of E1A. Also, while the adenovirus may carry a mutation anywhere in the E1B gene, it is specifically contemplated that mutations in the E1B gene that affect the ability of the E1B55 kD polypeptide to specifically bind the p53 polypeptide are part of the present invention. Thus, mutations contemplated include, but are not limited to, a mutation in the region encoding CR1 and/or CR2 of E1A and E1B55 kD protein of E1B. Any mutation in the E1A or E1B gene may be a deletion, insertion, or substitution. Furthermore, these mutations may be point mutants or they may involve multiple nucleotides. In addition, they may introduce a stop codon or a frame-shift, or both, into the coding sequence. In some embodiments of the invention, the mutation is a deletion, including a deletion encompassing residues 122-129 of the E1A amino acid sequence (SEQ ID NO:2). In other embodiments, the mutation may be a deletion encompassing nucleotides 2426 to 3328 of GenBank accession number NC_{—}001406, which is specifically incorporated by reference and which corresponds with nucleotides 185 to 1087 of the E1B nucleic acid sequence listed in SEQ ID NO:3. The E1B gene of SEQ ID NO:3 encodes an E1B55 kD protein (SEQ ID NO:4).

The adenoviruses of the invention are, in some embodiments, replication competent, meaning the virus is capable of replicating in a host cell. An adenovirus that is able to replicate only in the presence of a helper cell—a cell that provides complementing viral sequences necessary for the generation of viral particles—is not considered replication competent. Such an adenovirus is replication deficient.

The present invention is directed at cells with a mutated Rb, p53, or both Rb and p53 pathways. In some embodiments, the cell is a glial cell. In some cases, the cell, including a glial cell, is a tumor cell. Examples of non-glial cells that may form tumors in the cranium are neurons, ependiamal, meningeal and endothelial cells. Neurons can form neurocytomas and neuroblastomas. Other cells in a patient's cranium may be targeted by compositions and methods of the invention. Ependimal cells can form ependymomas. Glial and neuron precursor cells can form primitive neuroectodermic tumors (PNET) including medulloblastomas. Tumor cells that have metastasized to a patient's skull cavity are specifically contemplated as targets of the present oncolytic viruses and methods of treatment. These tumor cells may be from the following cancers: melanoma, non-small cell lung, small-cell lung, lung, hepatocarcinoma, retinoblastoma, gum, tongue, leukemia, CNS, head, neck, breast, pancreatic, prostate, renal, bone, testicular, ovarian, mesothelioma, cervical, gastrointestinal, lymphoma, colon, sarcoma or bladder.

Because compositions of the invention are contemplated for in vitro, ex vivo, and in vivo use on cells, some embodiments of the invention involve an adenovirus suitably dispersed in a pharmacologically acceptable formulation. The adenovirus may be in a composition, which may include a buffer and/or lipid. In some embodiments, the adenovirus is comprised in a lipid-based composition.

Methods of the invention involve administering compositions of the invention intracranially—within the cranium—to a patient. Administration may occur multiple times; in some cases, administration may be administered at least three times to the patient. In some embodiments, a composition is directly injected into a tumor in the cranium, while in other embodiments, administration is by perfusion. It is also contemplated that the adenoviral composition of the invention may be administered both intracranially and intravenously.

In some embodiments, the adenoviral sequence comprises a heterologous sequence. “Heterologous” sequence refers to a sequence that is heterologous with respect to adenovirus, and thus, it refers to a non-adenoviral sequence. The heterologous sequence or sequences may modify the activity of the adenovirus to effect a therapeutic result. In some embodiments, the heterologous sequence encodes a peptide sequence that can enhance the ability of the adenovirus to infect a cell. A heterologous sequence may encode an RGD motif, for example, or a regulatory sequence that may alter the expression of a particular sequence. Alternatively, a heterologous sequence may be a therapeutic polynucleotide that encodes a polypeptide with a therapeutic value. In some embodiments, the sequence encodes an enzyme that converts a pro-drug into an active chemotherapy drug; alternatively, the sequence may encode a protein that reduces or eliminates side effects from other cancer treatments such as chemotherapeutics, radiotherapeutics, or hormone therapy. The sequence may also encode a polypeptide that effects a cancer treatment itself, such as a tumor suppressor, or a protein that reduces metastasis, invasion, angiogenesis, or the incidence of secondary cancer. This sequence may also encode a RNA message or protein that will work to reduce or eliminate a pro-cancer signal such as an oncogene, angiogenesis, metastasis or invasion.

The compositions of the present invention may also be used to evaluate whether a cell has a mutation or defect in its Rb pathway or it may be used to study further adenoviruses as a therapeutic means. In some embodiments, the heterologous sequence encodes a reporter polypeptide.

Methods of the present invention include, in some embodiments, administering a second cancer therapy to a patient. The second therapy may be chemotherapy, immunotherapy, surgery, radiotherapy, immunosuppressive agents, or gene therapy with a therapeutic polynucleotide. It is contemplated that the second therapy is administered before, during, or after administration of compositions containing an adenovirus with a mutated E1A protein that is unable to bind Rb or an adenovirus with a mutated E1A protein and a mutated E1B region.

Chemotherapy is the second therapy in some methods of the invention. Chemotherapy may involve an alkylating agent, mitotic inhibitor, antibiotic, or antimetabolite. In other embodiments, it involves CPT-11, temozolomide, or a platin compound. In other methods of the invention, radiotherapy is the second therapy. It may involve X-ray irradiation, UV-irradiation, γ-irradiation, or microwaves. Alternatively, gene therapy may be employed as a second therapy. This therapy may involve providing a therapeutic polynucleotide encoding a therapeutic polypeptide, such as a tumor suppressor (but not Rb). It is further contemplated that more than one second therapy may be implemented in methods of the invention.

Dosages to be administered would be similar to those employed in gene therapy protocols involving viral vectors. In some embodiments, about 10³ to about 10¹⁵ viral particles are administered to the patient, while in others about 10⁵ to about 10¹² viral particles are administered to the patient. In other cases, about 10⁷ to about 10¹⁰ viral particles are administered to the patient.

Other steps that may be employed in the methods of the invention include: identifying a patient having a brain tumor; determining whether a cancer or tumor cell in a patient has a defective or mutated Rb pathway or has a mutation in Rb or an Rb pathway polypeptide; determining whether a patient is at risk for metastasis, determining whether a tumor has metastasized to the brain; and determining that a cell in a tumor does not have Rb inhibition of E2F-activation.

In other embodiments, the invention concerns a method for inhibiting the cell cycle progression of a glial cancer cell involving contacting the cell with an effective amount of a replication-competent adenovirus comprising an E1A polypeptide unable to bind Rb, wherein the amount is effective to inhibit the cell cycle progression of the cell.

Embodiments discussed in the context of a methods and/or composition of the invention may be employed with respect to any other method or composition described herein. Thus, an embodiment pertaining to one method may be applied to other methods of the invention as well.

“A” or “an,” as used herein in the specification, may mean one or more than one. As used herein in the claim(s), when used in conjunction with the word “comprising,” the words “a” or “an” may mean one or more than one.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

The following drawings form part of the present specification and are included to further demonstrate certain aspects of the present invention. The invention may be better understood by reference to one or more of these drawings in combination with the detailed description of specific embodiments presented herein:

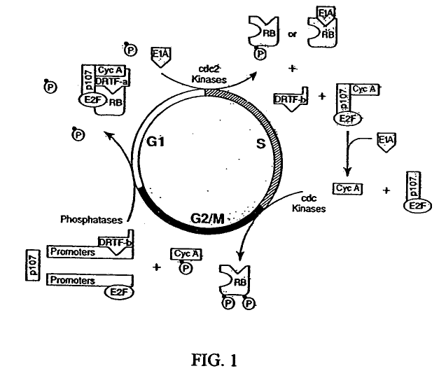

FIG. 1. Diagram of the Rb pathway (Newsham et al., 1998).

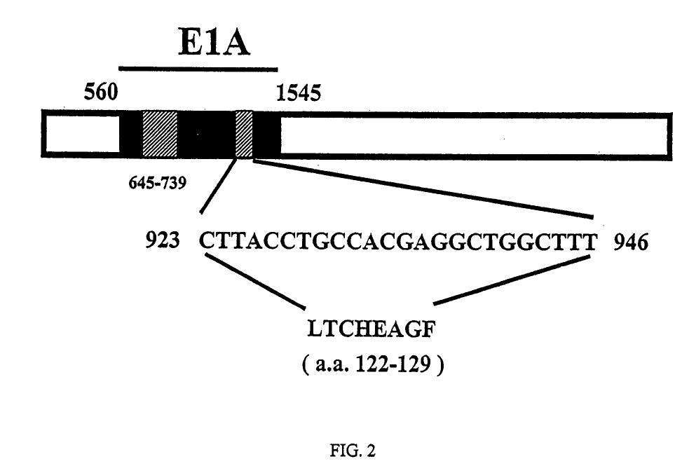

FIG. 2. Schematic representation of the D24 adenovirus. Shown are the 2 Rb-binding regions of the E1A sequence (hatched boxes). The 24 nucleotides that have been deleted and the corresponding amino acid translation are indicated.

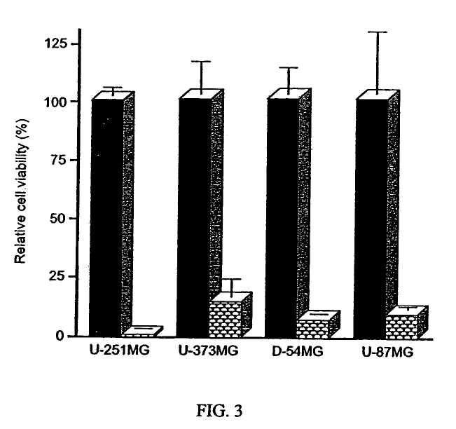

FIG. 3. Anti-cancer effects of the Delta24 adenovirus in vitro. Viability assay. Glioma cells were seeded in six-well culture plates for 20 h, and then infected with either Delta24 or UV-Delta24 adenovirus at 10 plaque forming units (PFU)/cell. Cells were counted in triplicate dishes of each treatment 7 (U-251 MG, U-373 MG), 9 (D-54 MG) or 14 (U-87 MG) days after infection, and the viability assessed by Trypan blue exclusion. Shown is the growth of cells treated with D24 (hatched bars) compared with control cells (100%). The number of viable cells is the mean±standard deviation (SD) of three different wells.

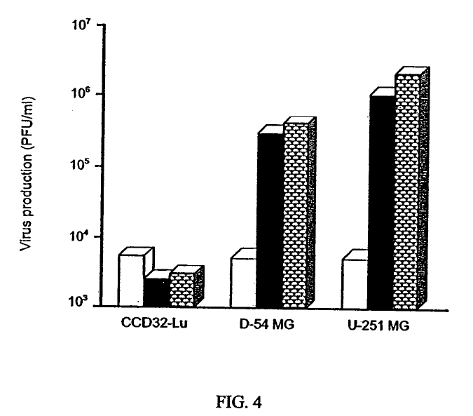

FIG. 4. Replication assays. Viral titration. Normal lung fibroblasts and D-54 MG and U-251 MG glioma cells were seeded at 104 cells per well in six-well plates and cultured with medium with 1% fetal bovine serum for 24 h and then infected with 10 PFU/cell of D24 adenovirus (empty bar). Six days after infection, the virus was released from the cells by 3 freeze/thaw cycles, and the resulting supernatant was titered on 293 cells. Virus yields are expressed as PFU/ml. Shown are the results from two independent experiments (solid and hatched bars).

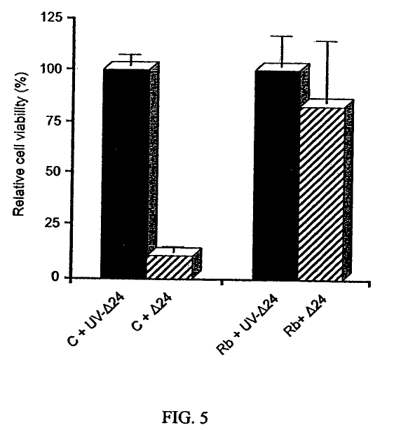

FIG. 5. Rescue of cancer cells from the oncolytic effect of Delta24 by Rb. Saos-2 cells were infected with 100 MOI of Ad5CMV-pA or Ad5CMV-Rb and 3 days later with 10 MOI of the Delta24 (solid bars) or UV-Delta24 (hatched bars). Five days after infection viability was assessed by Trypan blue exclusion. Shown is the relative growth of the control cells (100%) with respect to the Delta24 treated cells.

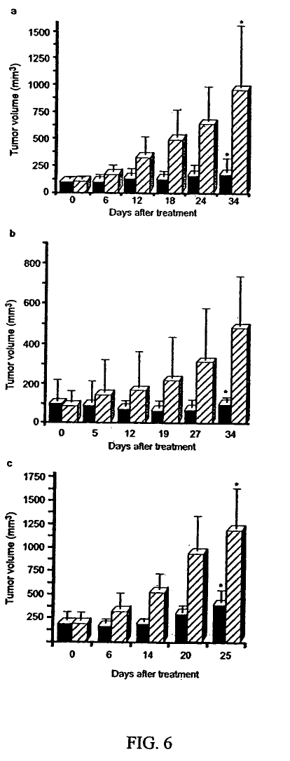

FIG. 6A.-6C. Anti-cancer effect in vivo. FIG. 6A. Mice with subcutaneous D-54 MG tumors (derived from a pre-established tumor line) were treated with 5×10⁸ PFU of the Delta24 (solid bars) or UV-D24 (hatched bars) given every 5 days a total of 5 times. Results are reported as the mean±SD. FIG. 6B. Mice with D-54 MG tumors (derived from inoculation of glioma cells) were treated with 10⁹ PFU of the Delta24 (solid bars) or UV-Delta24 (hatched bars) given every 5-7 days a total of 4 times. Results are reported as the mean±SD. FIG. 6C. Mice with tumors of D-54 MG glioma cells were treated with a single intratumoral injection of 10⁷ PFU of Delta24 (solid bars) or UV-Delta24 (hatched bars). Results are reported as the mean ±SD.

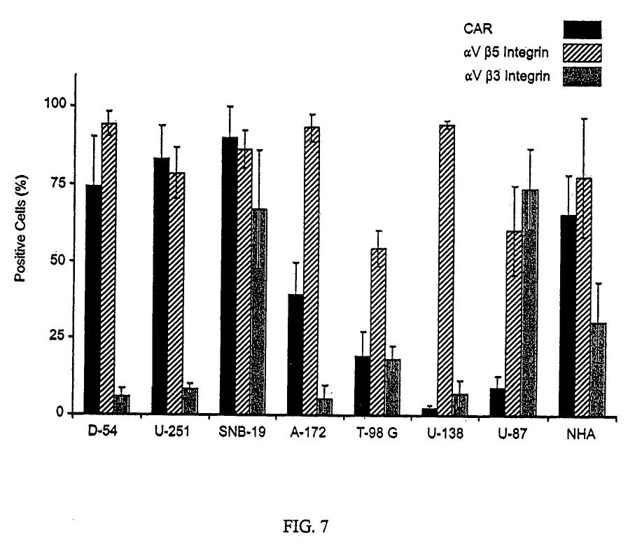

FIG. 7. Expression of coxsackie/adenovirus receptor (CAR) and integrins by D-54, U-251, SNB-19, A-172, T-98 G, U-138, U-87 and NHA.

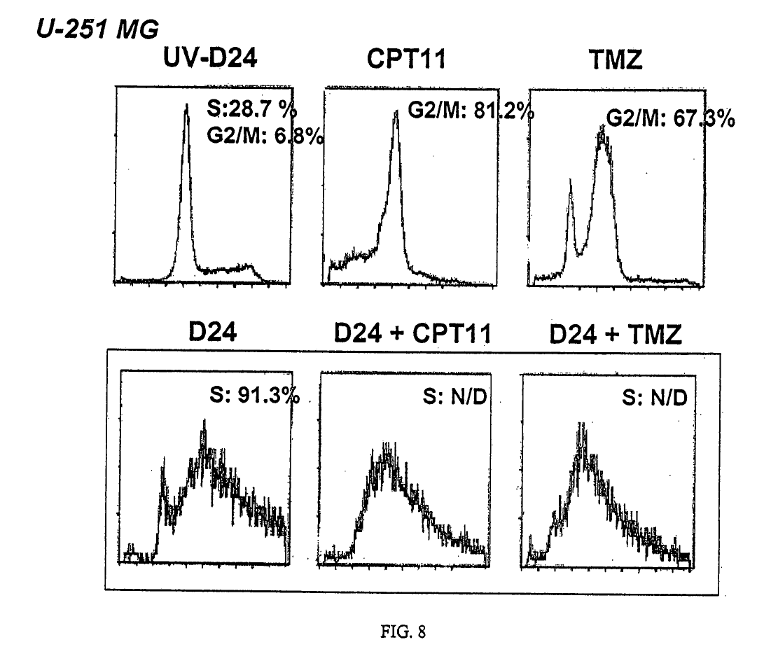

FIG. 8. Effect of the combination of Delta24 adenovirus and CPT-11 or TMZ on cell cycle progression analyzed by comparison of the cell cycle profiles of U-251 MG cells treated with Delta24 or UV-inactivated Delta24 after treatment with no drug, with CPT-1, or TMZ. Each drug was administered at a concentration corresponding to the IC₅₀ dose in U-251 MG cells. Representative cell cycle profiles 48 h post-treatment.

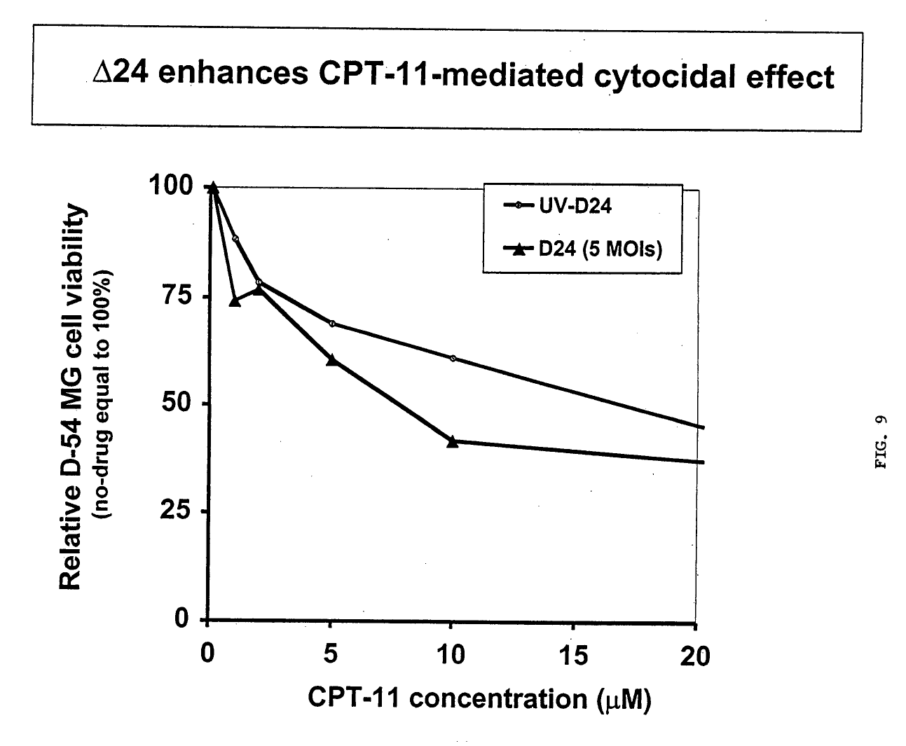

FIG. 9. Cell viability assessed by MTT assay. CPT-11-mediated anticancer effect was more prominent in cells that had been pretreated with Delta24.

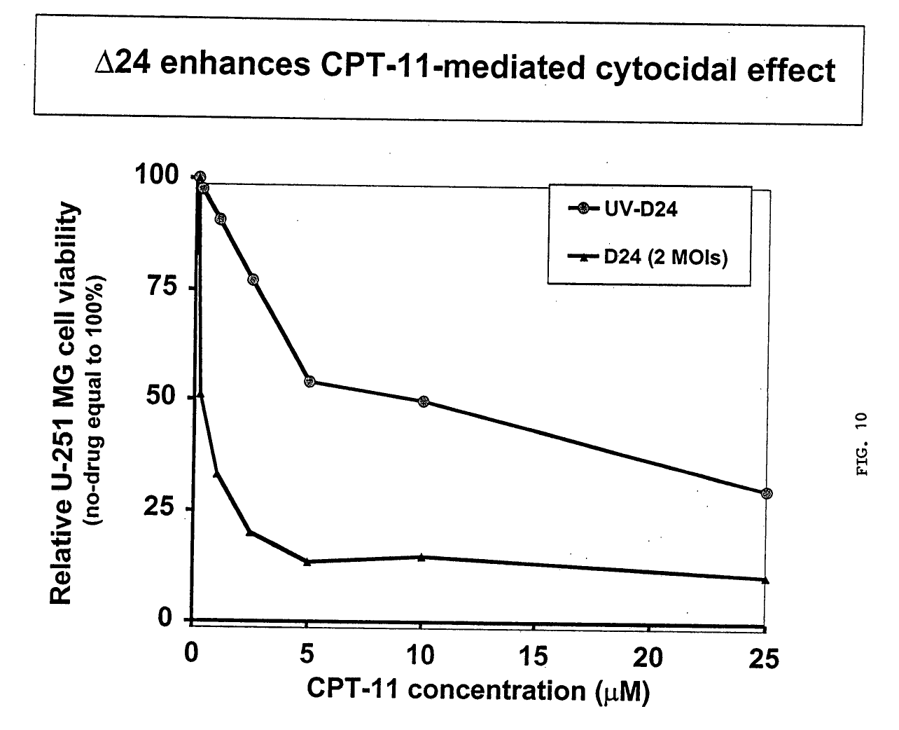

FIG. 10. Cell viability assessed by MTT assay. CPT-11-mediated anticancer effect was more prominent in cells that had been pretreated with Delta24.

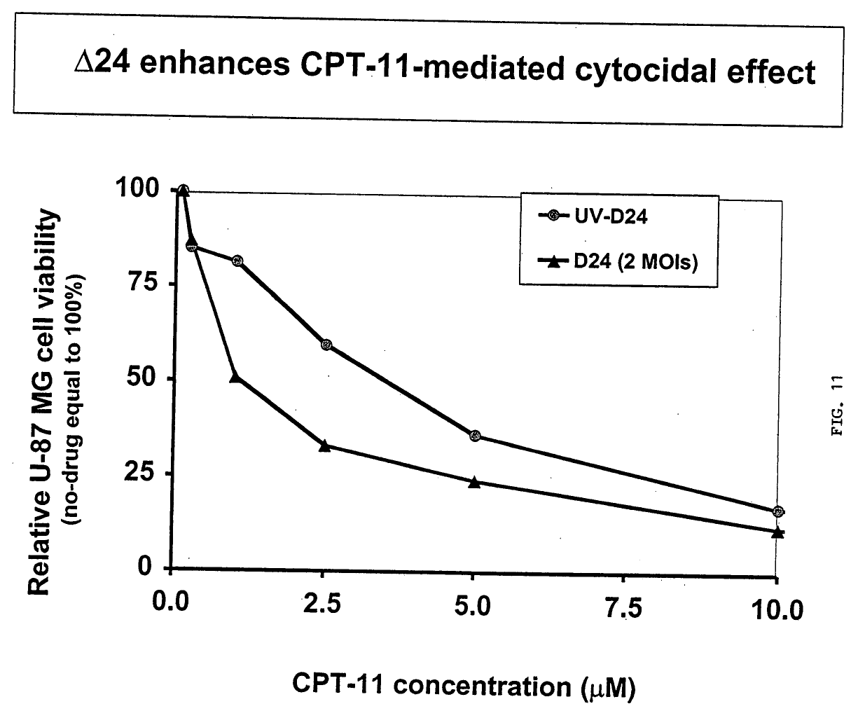

FIG. 11. Cell viability assessed by MTT assay. CPT-11-mediated anticancer effect was more prominent in cells that had been pretreated with Delta24.

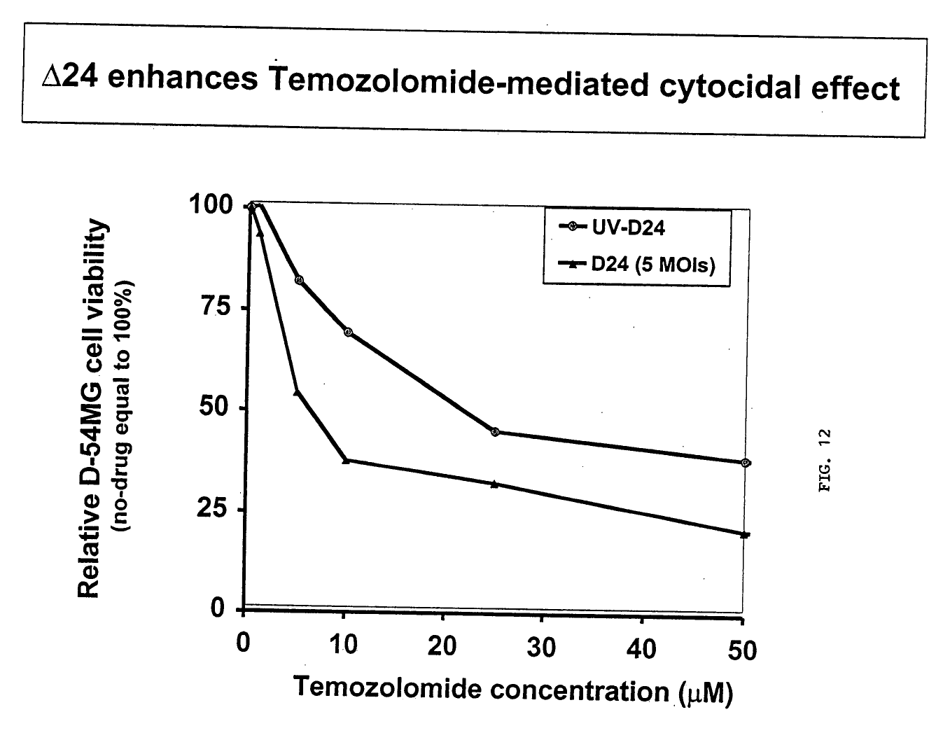

FIG. 12. Cell viability assessed by MTT assay. TMZ-mediated anticancer effect was more prominent in cells that had been pretreated with Delta24.

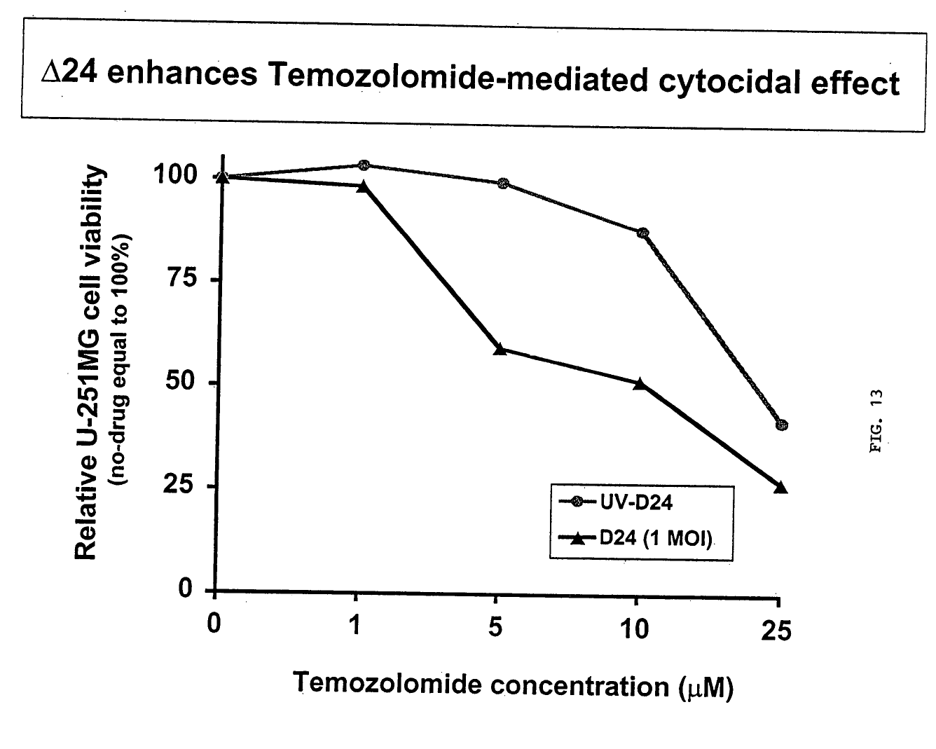

FIG. 13. Cell viability assessed by MTT assay. TMZ-mediated anticancer effect was more prominent in cells that had been pretreated with Delta24.

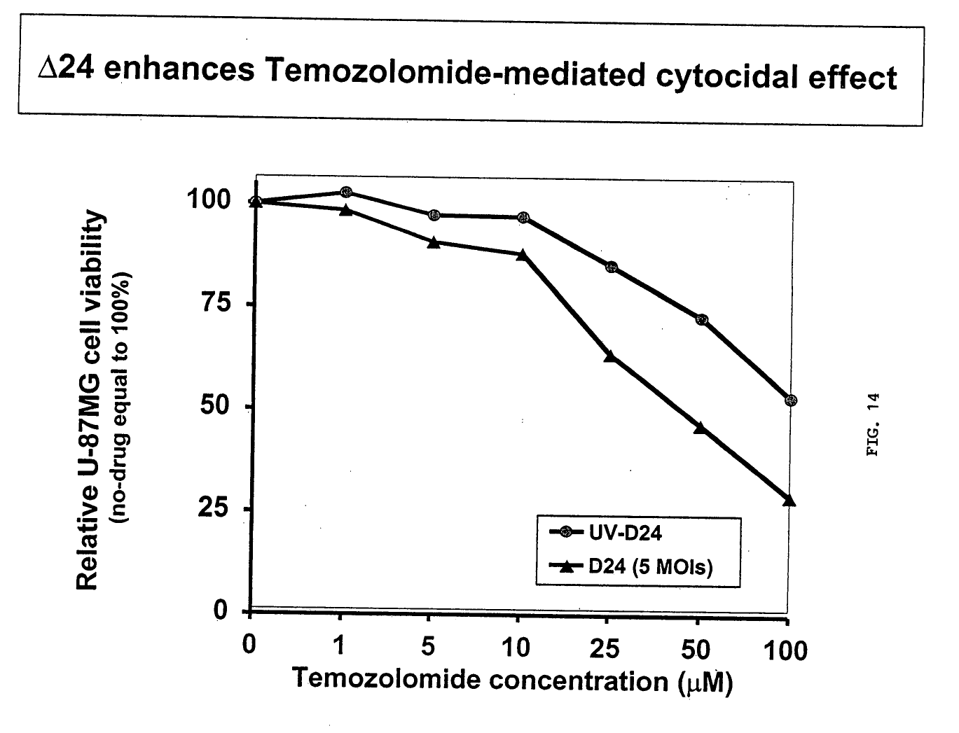

FIG. 14. Cell viability assessed by MTT assay. TMZ-mediated anticancer effect was more prominent in cells that had been pretreated with Delta24.

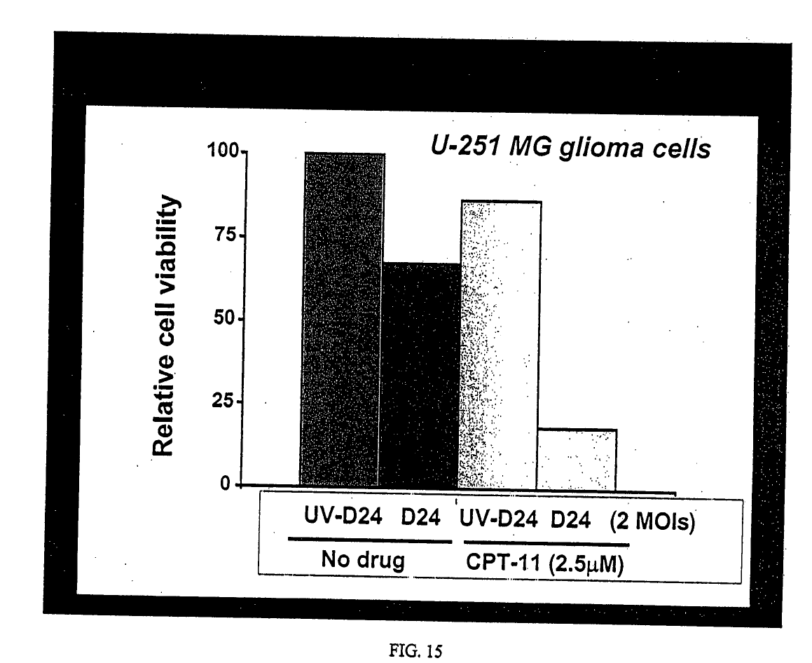

FIG. 15. Viability assay assessed by MTT of the Delta-24 (compared to UV-inactivated Delta-24), with or without CPT-11 at concentrations lower than the IC50. Synergistic effect was observed with the combination of the oncolytic and chemotherapeutic agents.

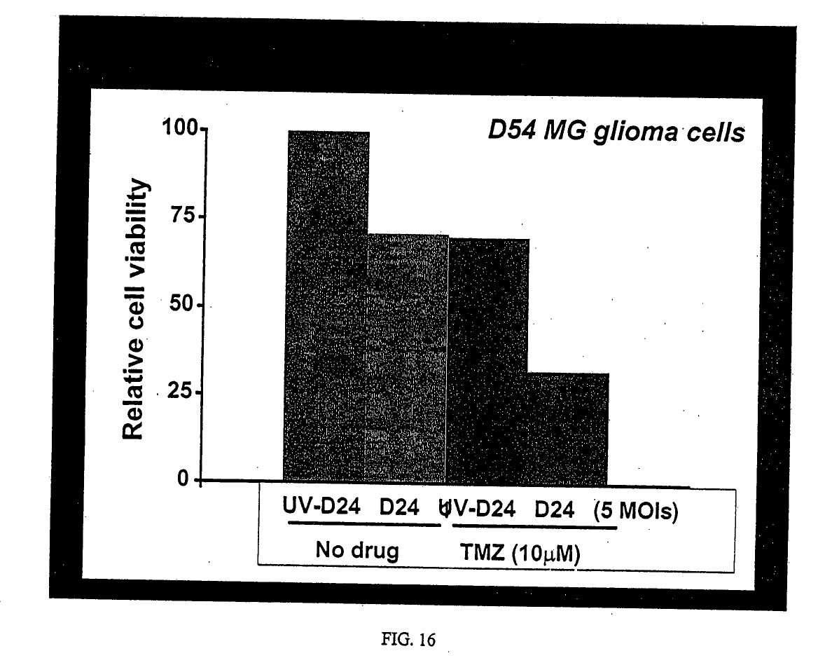

FIG. 16. Viability assay assessed by MTT of the Delta24 (compared to UV-inactivated Delta24), with or without TMZ at concentrations lower than the IC50. Synergistic effect was observed with the combination of the oncolytic and chemotherapeutic agents.

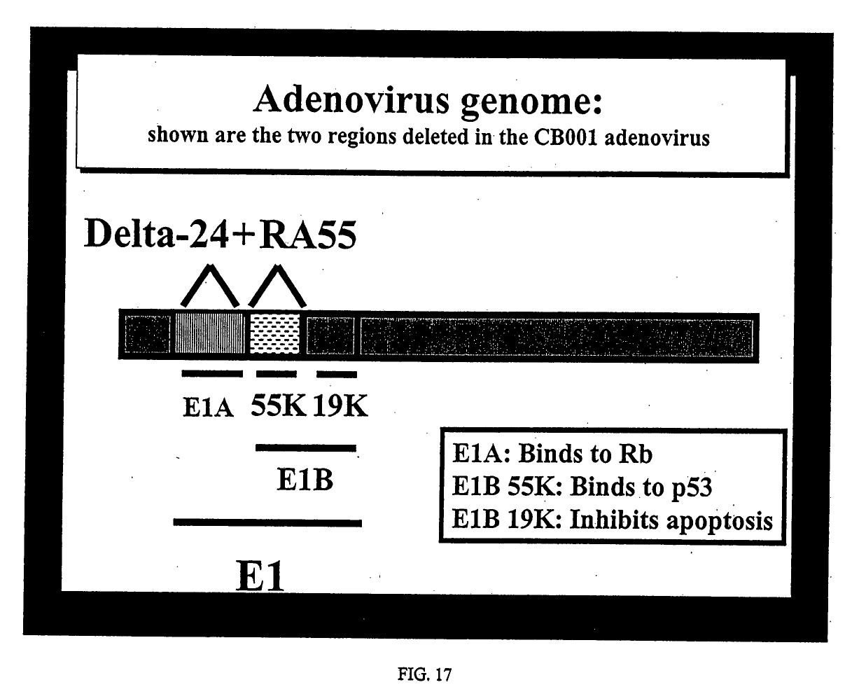

FIG. 17. Schematic of a double mutant adenovirus CB001 comprising partial deletions of the E1A and E1B adenoviral genes.

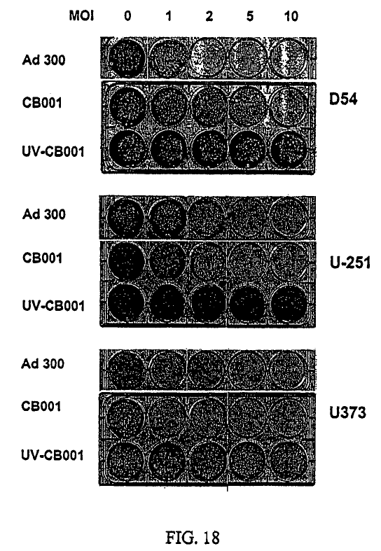

FIG. 18. An exemplary MTT assay for the analysis of CB001 mutants ability to induce cytopathic effect in human malignant glioma cell lines (D-54 MG, U-373 MG, U-251 MG). Dose dependence experiments showed that the cytopathic effect is highly reproducible and dose dependent in the three cell lines tested.

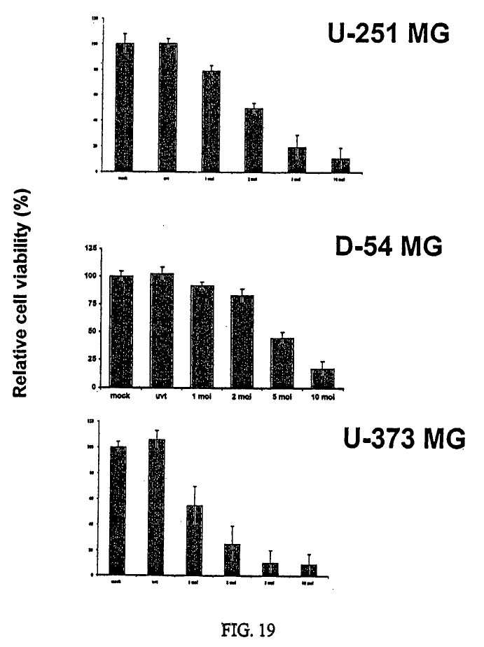

FIG. 19. crystal violet staining for analysis of CB001-induced cytopathic effect (CPE). CB001 exhibited increasing cytopathic effect with increasing MOIs in the cell lines tested.

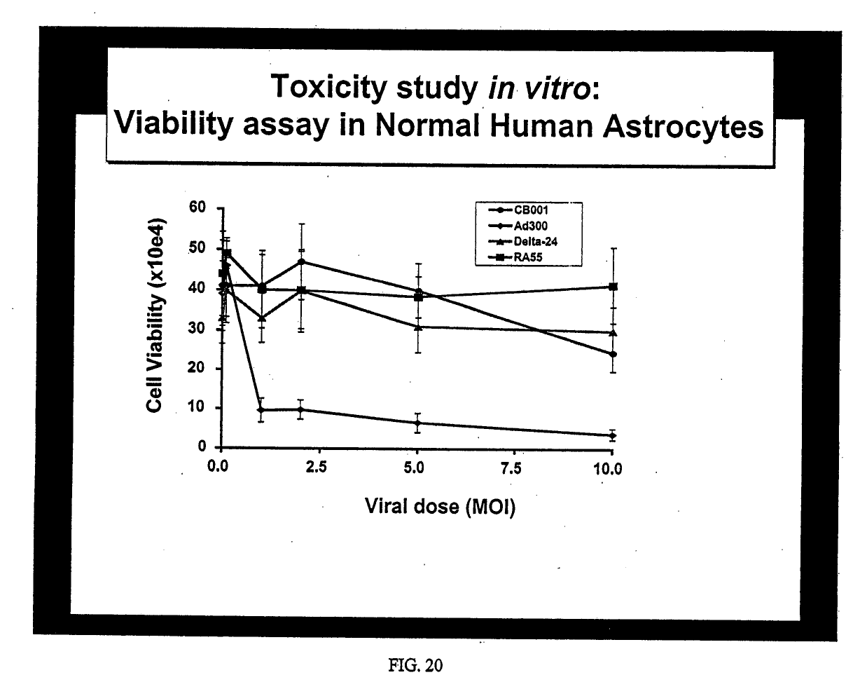

FIG. 20. Optical microscopy for analysis of CB001 replication phenotype in normal human astrocytes, cultures were grown as monolayers and arrested by serum starvation. Cells were then infected with CB001, Delta24, RA55, Ad300 or UV-inactivated-CB001 (control) adenovirus in a dose-dependent experiment. CB001 infection did not change the morphology of astrocytes as assessed by microscopy, and did not affect their viability ten days after the infection.

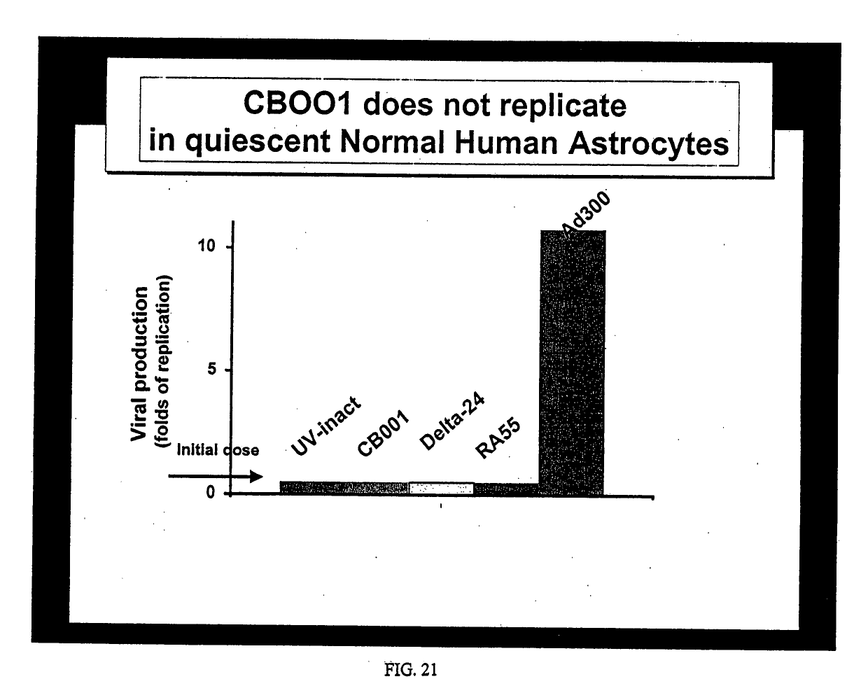

FIG. 21. Virus yields were determined by titration in 293 cells. Mormal Human Astrocytes (NHA) were infected with one MOI of either the CB001, Delta24, RA55 adenoviruses or the UV-inactivated adenoviruses or Ad300 wt control adenovirus. The Ad300 titer was 10.75 higher than the initial dose (normalized to 1) from three independent experiments. By contrast, the initial titer did not increase when the lysates were from CB001-, Delta24-, or RA55-treated NHA. The UV-Delta 24 infected cells did not induce production of new virus.

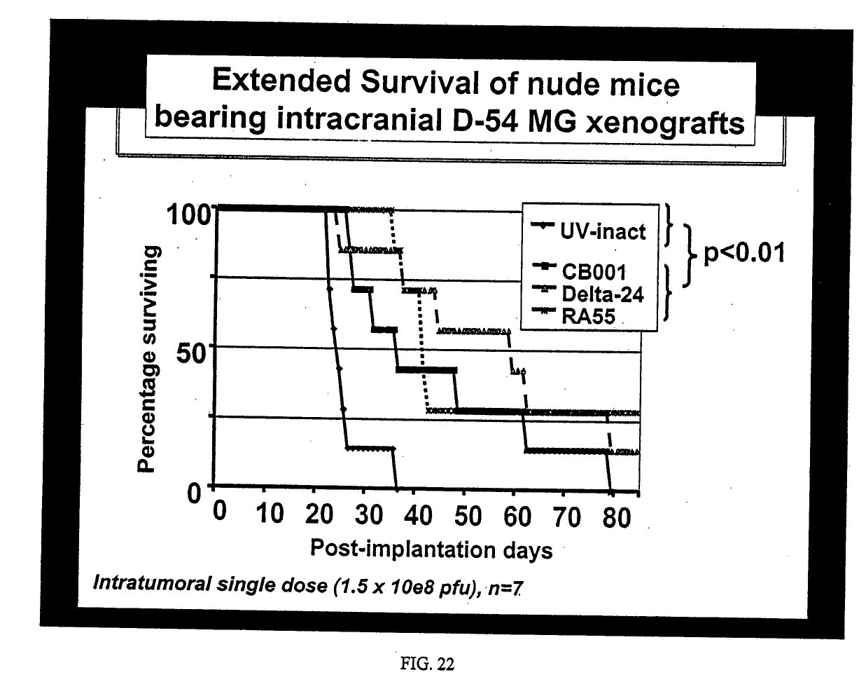

FIG. 22. Animal studies: Tumors were directly injected used a screw-guided surgical model with a single dose of 1.5×10⁸ pfu/tumor of CsCl-purified CB001, RA55, Delta24 or ultraviolet (UV)-inactivated Ad300 virus, as a negative control. After treatment, animal were observed for signs of toxicity and survival. Treatment of D-54 MG tumors with CB001 resulted in significant improvement of animal survival as compared with UV-inactivated Ad300 (p<0.01).

### DESCRIPTION OF ILLUSTRATIVE EMBODIMENTS

Prior to the present invention, the in vivo utility of oncolytic viruses was reduced or limited by the lack of cell-specific infection and the presence of infection-resistant cells within the tumor. The present invention comprises a method of treating cancer in a patient by administering a replication-competent mutant adenovirus (also referred to as an oncolytic adenovirus or a mutant oncolytic adenovirus) that is able to selectively kill tumor cells but does not affect cells with a functional retinoblastoma (Rb) and/or p53 pathways, due to an inability of the virus to modulate the pathway(s). Thus, cells with an intact Rb and/or p53 pathway may inhibit the production of the virus. In one embodiment, the mutant adenovirus comprises an E1A protein with a deletion of amino acids 122-129 (CR2), thus allowing replication of the adenovirus in select target cells with a defective Rb pathway. Any mutation in the adenovirus that reduces or eliminates the ability of the virus to modulate the Rb protein or any protein in the Rb pathway, so that transcription of the viral proteins occurs preferentially in Rb pathway defective cells, is contemplated. In other embodiments of the invention, mutations in the adenoviral E1A gene may be used in combination with mutations in the adenoviral E1B gene, in particular mutations in the E1B55K encoding gene. Such double-mutant adenovirus may further restrict adenoviral replication to a hyperproliferative cell.

The use of conditionally replicating adenoviruses for the treatment of human gliomas is especially attractive due to the intrinsic characteristics of brain tumors and the surrounding cell milieu. A double-mutant oncolytic adenovirus (CB001) was constructed encompassing a 24 bp-deletion in the Rb-binding region the E1A protein and a deletion the E1B gene that prevents the expression of the p53-binding E1B55 kDa protein. The double mutant, which is a double deletion, rendered CB001 unable to acquire a consistent replication phenotype in non-cycling astrocytes. Furthermore, the replication phenotype of CB001 in cycling astrocytes was more attenuated than those of Ad300 (wild type adenovirus). In vivo experiments show that treatment of human glioma xenografts with a single dose of 1.5×10⁸ pfu/tumor resulted in significant improvement of the percentage of tumor-bearing-animal survival. Pathologic examination of the tumor samples showed that CB001 was able to replicate in vivo as confirmed by the expression of late adenoviral genes. Taken collectively the exemplary data presented herein indicates that CB001 is a promising new anticancer agent with potentially lower toxicity that first generation oncolytic adenoviruses.

I. RB Pathway

Rb is a tumor suppressor gene whose loss of function is associated with tumor formation. While retinoblastomas are rare, cancers involving the retinoblastoma protein (Rb) are not. Included are gliomas, sarcomas, tumors of the lung, breast, ovary, cervix, pancreas, stomach, colon, skin, larynx, bladder and prostate. Unphosphorylated Rb acts as a tumor suppressor. Rb inhibits cell proliferation by arresting cells in G₁ of the cell cycle. Upon phosphorylation of Rb, transcriptional factors, such as E2F, are released. The binding of E1A causes transcriptional factor release in much the same manner as phosphorylation. Several viral oncoproteins target Rb for inactivation in order to facilitate viral replication. These proteins include adenovirus E1A, SV40 large T antigen, and papillomavirus E7.

The E1A protein is one of the first virus-specific polypeptides synthesized after adenoviral infection and is required for viral replication to occur (Dyson and Harlow, 1992; Flint and Shenk, 1997). The targets of E1A proteins, such as Rb, modulate the cell cycle by regulating cell progression from G₀ and G₁ into S phase. In the case of Rb, binding between the Rb protein and the E1A protein results in release of E2F from pre-existing cellular E2F-Rb complexes. E2F is then free to activate both the E2 promoter of the adenovirus and several cell cycle-regulatory genes of the infected cell. The transcriptional activation of these cellular genes in turn helps to create an environment suitable for viral DNA synthesis in otherwise quiescent cells (Nevins, 1992). Two segments of E1A are important for binding Rb; one includes amino acids 30-60 and the other amino acids 120-127 (Whyte et al., 1988; Whyte et al., 1989). Deletion of either region prevents the formation of detectable E1A/Rb complexes in vitro and in vivo (Whyte et al., 1989).

An adenovirus containing a Delta24 (Δ24) mutation, which is a deletion of nucleotides encoding amino acid 122-129 of E1A, produces an E1A protein that cannot bind Rb, causing an infected cell to remain in G₀. In this instance, there is no adenoviral transcription. E2F activity will allow adenoviral transcription to occur. Thus a mutant Rb pathway and a mutant E1A, along with E2F activation are necessary for delta24 adenoviral transcription. Thus, a functional retinoblastoma pathway will typically protect a cell from adenoviral-mediated cell death.

Retinoblastoma or Rb, as used herein, refers to the polypeptide encoded by the retinoblastoma gene. The retinoblastoma gene is found at chromosomal location 13q14 and encodes a protein of approximately 110 kiloDaltons (kD).

Retinoblastoma (Rb) pathway, as used herein, refers the interaction of a group of regulatory proteins that interact with the Rb protein or other proteins that interact with Rb, either upstream or downstream of Rb, in regulating cell proliferation (FIG. 1).

Proteins within the Rb pathway include, but are not limited to, Rb, the E2F family of transcription factors, DRTF, RIZ286, MyoD287, c-Abl288, MDM2289, hBRG1/hBRM, p16, p107, p130, c-Abl tyrosine kinase and proteins with conserved LXCXE motifs, cyclin E-cdk 2, and cyclin D-cdk 4/6. Phosphorylation of Rb by cyclin D and cyclin E-associated kinases releases E2F, which is bound to unphosphorylated Rb. E2F then stimulates cyclin E transcription. Increased cyclin E-cdk2 kinase activity results in more Rb phosphorylation and greater E2F. Unphosphorylated Rb acts as a tumor suppressor by binding to regulatory proteins that increase DNA replicaiton, such as E2F (The Genetic Basis of Human Cancer, Vogelstein and Kinzler eds., 1998).

Defective retinoblastoma pathway, as used herein, refers to inactivation, mutation, or deletion of the Rb protein or the inability of the upstream or downstream regulatory proteins that interact with the Rb to regulate cell proliferation due to a mutation or modification of one or more proteins, protein activities, or protein-protein interactions. The present invention includes compositions and methods that may be administered to a cell with a defective Rb pathway, which includes the inability of Rb to repress the transcription-activating activity of E2F. E2F activates transcription of cellular genes and adenoviral DNA if its activity is not repressed. Examples of ways in which E2F could escape repression include, but are not limited to, Rb not being able to bind E2F (i.e. E1A binding to Rb), overexpression of E2F, a low amount of Rb and situations in which Rb remains phosphorylated.

Mutation causing a defective Rb pathway include, but is not limited to inactivating mutations in Rb, INK4 proteins, and CIP/KIP and activating mutations in the cyclin genes, such as cyclin D/cdk 4, 6 and cyclin E, cdk 2. Mutations in one or another element of the Rb regulatory pathway, including p16, cyclin D, cdk4, E2F or Rb itself, may be mutated in almost 100 percent of human tumors (The Genetic Basis of Human Cancer, 1998).

A number of DNA tumor viruses encode proteins that are capable of interacting with Rb and causing cellular transformation. Viral oncoproteins are encoded by adenoviruses (Ad-2 and Ad-5), polyomaviruses (SV40), and papillomaviruses (HPV-16). Adenoviruses encode E1A, SV40 encodes the large T oncoprotein, and HPV-16 encodes E7 (The Genetic Basis of Human Cancer, 1998). Mutations in any of these proteins that cause an inability to bind to Rb are contemplated to be effective in the present invention.

II. Adenovirus

The present invention comprises a genetically engineered form of adenovirus. Adenovirus is a 36 kb, linear, double-stranded DNA virus (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Advantages of adenovirus include a mid-sized genome, ease of manipulation, high titer, a wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 map units (m.u.)) is particularly efficient during the late phase of infection, and all the mRNAs issued from this promoter possess a 5′-tripartite leader (TPL) sequence, which makes them preferred mRNAs for translation.

Adenovirus of the present invention may be further modified to contain an expression cassette containing a heterologous gene. In one embodiment, the delta24 adenovirus, described herein, may be further modified to contain an expression cassette containing a heterologous gene. In other embodiments, a double mutant (e.g., delta24/55 kDa⁻ adenovirus also designated CB001, described herein) may be further modified to contain an expression cassette containing a heterologous gene. Examples of such heterologous genes include cytosine deaminase (to convert 5-FC to 5-FU), anti-sense VEGF, Bcl-2, or interferons alpha, beta or gamma. The development of viral vectors was an improvement to the field of gene transfer, as demonstrated by U.S. Patent Application Ser. No. 60/078,205, hereby incorporated by reference. The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). Adenovirus can be readily introduced in a variety of cell lines and laboratory animals (Nicolas and Rubenstein, 1988; Temin, 1986).

A. Oncolytic Adenovirus

Adenoviruses that have a mutated E1A and/or E1B, which are unable to bind Rb or p53, respectively. These oncolytic adenoviruses of the invention may be used against cancer cells with a mutated Rb, p53, or both Rb and p53 proteins or pathways. The mutations may be in any location that causes the E1A or E1B proteins to be unable to bind Rb or p53 respectively. The tumor selective ability of the present invention is dependent upon the cell having a defective Rb and/or p53 pathway, which may allow expression of viral proteins and replication of the virus resulting in lysis of the target cells. Also included in the present invention is any mutation in the adenoviral genome that inhibits adenoviral replication in non-tumor cells and allows conditional replication in hyperproliferative cells.

Wild type adenovirus E1A typically induces cell proliferation. E1A activity is dependent upon simultaneous expression of the E1B gene to circumvent apoptosis and in particular the E1B55kD protein, which binds and modulates the p53 protein. Two segments of E1A are important for binding Rb. One segment includes amino acids 30-60 (CR1) and the other segment includes amino acids 120-127 (CR2) (Whyte et al., 1988; Whyte et al., 1989). Deletion of either segment prevented the formation of detectable E1A/Rb complexes in vitro and in vivo (Whyte et al., 1989). Delta24 adenovirus is a mutant adenovirus that encodes an E1A protein with deletion of amino acids 122-129 (CR2) that selectively targets cells with abnormal Rb control.

The mutant adenovirus delta24 is a potent cytolytic agent against glioma in vitro and in vivo. The presence of a functionally active Rb protein in quiescent normal cells halts the cytopathic effect of the delta24 adenovirus. Thus, the E1A-mutant protein was unable to deregulate the cell cycle and replicate in cells containing a wild-type Rb pathway. Transfer of exogenous Rb to Rb-null cells resulted in resistance to the adenoviral effect in previously virus-sensitive cancer cells, thus confirming the ability of the wild-type Rb protein to block delta24 replication. These results are important for therapeutic applications of the delta24 adenovirus because the existence of a cell-cycle-mediated mechanism of resistance to the virus suggests that the anticipated toxicity of an adenovirus expressing a handicapped E1A protein will be limited to the very few, occasionally dividing, reactive glial cells in the adult human brain.

The anti-cancer effect of the adenovirus is potent. In vitro, cell destruction is massive even with very low doses of the virus. In vivo, a single local injection of a low dose of the adenovirus resulted in subcutaneous tumor inhibition in the mouse tumor xenograft model. Multiple injections produced tumor regression in 4 of 11 mice. Regression of human gliomas established subcutaneously in mice is a strong indication of the power of the replication-competent strategy. In fact, the inability of current vectors to transduce sufficient numbers of tumor cells in vivo may be the major hurdle to successful gene therapy of cancer (Roth and Cristiano, 1997), especially in cases in which the bystander effect on nontransduced cells is limited. Thus, replication-deficient adenovirus could transfer an exogenous gene to less than 15% of glioma cells in human patients (Puumalainen et al., 1998).

Several factors favor the use of oncolytic adenoviruses for the treatment of brain tumors. First, gliomas do not metastasize, and therefore an efficient local approach should be enough to cure the disease. Second, every glioma harbors several populations of cells expressing different genetic abnormalities (Sidransky et al., 1992; Collins and James, 1993; Fumari et al., 1995; Kyritsis et al., 1996). Thus, the spectrum of tumors sensitive to the transfer of a single gene to cancer cells may be limited. Third, replication competent adenoviruses can infect and destroy cancer cells that are arrested in G₀. Since gliomas invariably include non-cycling cells, this property is important. Finally, the p16-Rb pathway is abnormal in the majority of gliomas (Hamel et al., 1993; Henson et al., 1994; Hirvonen et al., 1994; Jen et al., 1994; Schmidt et al., 1994; Costello et al., 1996; Fueyo et al., 1996b; Kyritsis et al., 1996; Ueki et al., 1996; Costello et al., 1997), thus making the delta24 strategy appropriate for most of these tumors. The loss of the retinoblastoma tumor suppressor gene function has been associated with the causes of various types of tumors.

In other embodiments of the invention, an E1A mutation (e.g., a delta24 mutation in E1A) may be used in combination with mutations in the E1B region of the same adenovirus, thus producing a double mutant adenovirus (e.g., CB001). In certain embodiments of the invention an adenovirus may comprise a deletion of 24 bp of the E1A region (delta24 mutation) and a deletion in the E1B region that prevents expression of the E1B55 kD protein. The E1B55 kD protein has been shown to bind to and inactivate p53. The E1B region mutation may include a deletion of adenovirus sequences from 2426 bp to 3328 bp of genebank accession number NC_{—}001406, which is incorporated herein by reference. The apoptosis suppressing E1B19 kD protein, also encoded by the E1B region, may still be expressed by an adenovirus with a deletion from 2426 bp to 3328 bp.

In certain embodiments of the invention, oncolytic adenovirus may be used as an adenovirus expression vector. One method for in vivo delivery of therapeutic genes or agents involves the use of an adenovirus expression vector. “Adenovirus expression vector” is meant to include those vectors containing adenovirus sequences sufficient to (a) support packaging of the vector and (b) to express a polynucleotide that has been cloned therein. In this context, expression may require that the gene product be synthesized.

The insertion position of a heterologous gene of interest within the adenovirus sequences is not critical to the invention. The polynucleotide encoding the gene of interest may be inserted in lieu of the deleted E3 region in E3 replacement vectors as described by Karlsson et al., (1986) or other region that are not essential for viral replication in the target cell.

If an adenovirus has been mutated so that it is unable to replicate or is conditionally replicative (replication-competent under certain conditions), a helper cell may be required for viral replication. When required, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, for exmple Vero cells or other monkey embryonic mesenchymal or epithelial cells. As preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI (multiplicity of infection) of 0.05. Cultures are left stationary overnight, following which the volume is increased to 100% and shaking commenced for another 72 h.

The nature of the adenovirus vector is not believed to be crucial to the successful practice of the invention. The adenovirus may be of any of the 42 different known serotypes or subgroups A-F. Adenovirus type 5 is the preferred starting material for use in the present invention. Adenovirus type 5 is a human adenovirus about which a great deal of biochemical and genetic information is known, and it has historically been used for most constructions employing adenovirus as a vector.

Adenovirus is easy to grow and manipulate and exhibits broad host range in vitro and in vivo. This group of viruses can be obtained in high titers (e.g., 10⁹-10¹¹ plaque-forming units (pfu) per ml), and they are highly infective. The life cycle of adenovirus does not require integration into the host cell genome. No side effects have been reported in studies of vaccination with wild-type adenovirus (Couch et al., 1963; Top et al., 1971), demonstrating their safety and therapeutic potential for use in treating cancer.

Adenovirus vectors have previously been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include tracheal instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993). Recent reports have shown that adenovirus are useful as tumor-selective oncolytic virus (Bischof et al., 1996; Heise et al., 1997; Freytag et al., 1998; Kim et al., 1998)

B. Methods for Producing Viral Particles

The traditional method for the generation of adenoviral particles is co-transfection followed by subsequent in vivo recombination of a shuttle plasmid (usually containing a small subset of the adenoviral genome and the gene of interest in an expression cassette) and an adenoviral helper plasmid (containing most of the entire adenoviral genome) into either 293 or 911 cells (obtained from Introgene, The Netherlands). In the present invention, the adenovirus is replication-competent in cells with a mutant Rb pathway. After transfection, the adenoviral plaques are isolated from the agarose overlaid cells and the viral particles are expanded for analysis. For detailed protocols the skilled artisan is referred to Graham and Prevac, 1991.

Alternative technologies for the generation of adenovirus or adenovirus expression vectors include utilization of the bacterial artificial chromosome (BAC) system, in vivo bacterial recombination in a recA+ bacterial strain utilizing two plasmids containing complementary adenoviral sequences, and the yeast artificial chromosome (YAC) system. PCT publications 95/27071 and 96/33280 provide details of adenoviral production methodologies and are herein incorporated by reference.

C. Disease States

Various embodiments of the present invention deals with the treatment of disease states comprised of cells that are deficient in the Rb and/or p53 pathway. In particular, the present invention is directed at the treatment of diseases, including but not limited to retinoblastomas, gliomas, sarcomas, tumors of lung, ovary, cervix, pancreas, stomach, colon, skin, larynx, breast, prostate and metastases thereof. The retinoblastoma gene has been implicated in breast tumorigenesis and may be inactivated in approximately 20 percent of breast cancers.

There are various categories of brain tumors. Glioblastoma multiforme is the most common malignant primary brain tumor of adults. More than half of these tumors have abnormalities in genes involved in cell cycle control. Often there is a deletion in the CDKN2A or a loss of expression of the retinoblasoma gene. Other types of brain tumors include astrocytomas, oligodendrogliomas, ependymomas, medulloblastomas, meningiomas and schwannomas.

D. Modifications of Oncolytic Adenovirus

Modifications of oncolytic adenovirus described herein (e.g., delta24 and CB001) may be made to improve the ability of the oncolytic adenovirus to treat cancer. The present invention also includes any modification of oncolytic adenovirus that improves the ability of the adenovirus to treat neoplastic cells. Included are modifications to oncolytic adenovirus genome in order to enhance the ability of the adenovirus to infect and replicate in cancer cells by altering the receptor binding molecules.

The absence or the presence of low levels of the coxsackievirus and adenovirus receptor (CAR) on several tumor types can limit the efficacy of the oncolytic adenovirus. Various motifs may be added to the fiber knob, for instance an RGD motif (RGD sequences mimic the normal ligands of cell surface integrins), Tat motif, poly-lysine motif, NGR motif, CTT motif, CNGRL motif, CPRECES motif or a strept-tag motif (Rouslahti and Rajotte, 2000). Peptide sequences that bind specific human glioma receptors such as EGFR or uPR may also be added. Specific receptors found exclusively or preferentially on the surface of cancer cells may also be a target for adenoviral binding and infection, such as EGFRvIII. A motif, such as RGD, can be inserted into the HI loop of the adenovirus fiber protein. Modifying the capsid allows the adenoviral construct to bind to integrins without binding CAR. The motifs allow CAR-independent target cell infection. This allows higher replication, more efficient infection, and increased lysis of tumor cells (Suzuki et al., 2001, incorporated herein by reference).

Modifications of an oncolytic adenovirus genome could also be made by inserting cassettes into the adenovirus genome to express foreign genes within tumor cells. Possible foreign genes include pro-drug converting enzymes, such as cytosine deaminase to convert non-toxic 5-FC into the active chemotherapy drug 5-FU. Anti-angiogenesis molecules such as anti-sense VEGF, dominant negative forms of angiogenesis-related receptors, inhibitors of metalloproteases, or a gene coding for endostatin or angiostatin may also be incorporated into an adenoviral expression vector of the present invention. Still other foreign genes include anti-apoptotic molecules such as Bcl-2 and immunomodulators such as interferon gamma, alpha, or beta, or interleukin molecules.

Modifications may be made to an oncolytic adenoviral genome in order to increase the ability of the adenovirus to escape from an anti-viral immunoresponse. For example, the negative charge of the adenoviral capsid may be modified.

Regulatory elements may be inserted into an oncolytic adenoviral genome in order to control temporal expression of adenoviral genes or target certain tissues or cells for adenoviral replication. Regulatory elements that could be inserted include GAFP-related promoters, prednisone-sensitive enhancers, the CEA promoter or E2F gene regulated control elements in conjunction with adenoviral or heterologous genes.

III. Methods for Treating Rb-Related Conditions

The present invention involves the treatment of neoplasms with at least a disrupted Rb pathway. The types of conditions that may be treated include conditions that involve cells with a defective Rb pathway. It is contemplated that a wide variety of tumors may be treated using the methods and compositions of the invention, including gliomas, sarcomas, and tumors of the lung, breast, prostate, or brain metastases.

In many contexts, it is not necessary that the cell be killed or induced to undergo cell death or “apoptosis.” Rather, to accomplish a meaningful treatment, all that is required is that the tumor growth be slowed to some degree. It may be that the cell's growth is completely blocked or that some tumor regression is achieved. Clinical terms such as “remission” and “reduction of tumor” burden also are contemplated given their normal usage.

The term “therapeutic benefit” refers to anything that promotes or enhances the well-being of the subject with respect to the medical treatment of his/her condition, which includes treatment of pre-cancer, cancer, and hyperproliferative diseases. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition.

A. Adenoviral Therapies

Those of skill in the art are well aware of how to apply adenoviral delivery to in vivo and ex vivo situations. For viral vectors, one generally will prepare a viral vector stock. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, or 1×10¹² infectious particles to the patient. Formulation as a pharmaceutically acceptable composition is discussed below.

Various routes are contemplated for various tumor types. The section below on routes contains an extensive list of possible routes. Where discrete tumor mass, or solid tumor, may be identified, a variety of direct, local and regional approaches may be taken. For example, the tumor may be directly injected with the adenovirus. A tumor bed may be treated prior to, during or after resection. Following resection, one generally will deliver the adenovirus by a catheter left in place following surgery. One may utilize the tumor vasculature to introduce the vector into the tumor by injecting a supporting vein or artery. A more distal blood supply route also may be utilized.

The method of treating cancer includes treatment of a tumor as well as treatment of the region near or around the tumor. In this application, the term “residual tumor site” indicates an area that is adjacent to a tumor. This area may include body cavities in which the tumor lies, as well as cells and tissue that are next to the tumor.

In some embodiments of the present invention a subject is exposed to a viral vector and the subject is then monitored for toxicity, where such toxicity may include, among other things, causing a condition that is injurious to the subject.

B. Formulations and Routes of Administration to Patients

Where clinical applications are contemplated, it will be necessary to prepare pharmaceutical compositions (expression vectors, virus stocks, proteins, antibodies and drugs) in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. The routes of administration will vary, naturally, with the location and nature of the lesion, and include, e.g., intradermal, transdermal, parenteral, intracranial, intravenous, intramuscular, intranasal, subcutaneous, percutaneous, intratracheal, intraperitoneal, intratumoral, perfusion, lavage, direct injection, and oral administration and formulation. In the present invention, intracranial or intravenous administration are preferred embodiments. Administration may be by injection or infusion. Please see Kruse et al. (J. Neuro-Oncol., 19:161-168, 1994), specifically incorporated by reference, for methods of performing intracranial administration. Such compositions would normally be administered as pharmaceutically acceptable compositions, described supra.

Intratumoral injection, or injection into the tumor vasculature is specifically contemplated for discrete, solid, accessible tumors. Local, regional or systemic administration also may be appropriate. For tumors 1.5 to 5 cm in diameter, the injection volume will be 1 to 3 cc, preferably 3 cc. For tumors greater than 5 cm in diameter, the injection volume will be 4 to 10 cc, preferably 5 cc. Multiple injections delivered as single dose comprise about 0.1 to about 0.5 ml volumes, preferable 0.2 ml. The viral particles may advantageously be contacted by administering multiple injections to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising the adenovirus. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catheterization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

The adenovirus also may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

In a further embodiment of the invention, the adenovirus may be delivered to cells using liposome or immunoliposome delivery. The adenovirus may be entrapped in a liposome or lipid formulation. Liposomes may be targeted to neoplasic cell by attaching antibodies to the liposome that bind specifically to a cell surface marker on the neoplastic cell. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

The production of lipid formulations often is accomplished by sonication or serial extrusion of liposomal mixtures after (I) reverse phase evaporation (II) dehydration-rehydration (111) detergent dialysis and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

A contemplated method for commercial scale preparation of a lipid composition is, generally speaking, mixing of the components, drying the mixture, re-hydrating the mixture, dispersing the mixture, extruding the lipid composition through filters of decreasing pore size and mixing of the lipid composition with a therapeutic agent to form a lipoplex. The following provides additional information about manufacturing and formulating a lipoplex for use in the delivery of an adenovirus to a cell.

Powdered components are weighed, mixed, and dissolved in an acceptable solvent, such as tertiary butanol, chloroform, methanol, ethylene chloride, ethanol, or mixtures of these solvents. It is contemplated that any two of the solvent are present in a ration of about 1:1000, 1:500, 1:100, 1:50, 1:25, 1:10, 1:5 or 1:1. Solublization with tertiary butanol may be employed, due to the carcinogenic properties of chloroform. Although, chloroform can be used if steps are taken to limit the residual chloroform present in the lipid composition to acceptable levels. It is envisioned that other lipophilic solvents may be used for the solubilization of the lipid components. The lipid mixture may be solubilized in tert-butanol at a temperature of about 0° C., 2° C., 4° C., 6° C., 8° C., 10° C., 12° C., 14° C., 16° C., 18° C., 20° C., 22° C., 24° C., 26° C., 28° C., 30° C., 32° C., 34° C., 36° C., 38° C., 40° C., 42° C., 44° C., 46° C., 48° C., 50° C., 52° C., 54° C., 56° C., 58° C., 60° C., 62° C., 64° C., 66° C., 68° C., 70° C., 72° C., 74° C., 76° C., 78° C., 80° C., 82° C., 84° C., 86° C., 88° C., or 90° C. A range of temperatures, such as 5° C. to 80° C., 10° C. to 70° C., 20° C. to 60° C., and 30° C. to 50° C., is also contemplated for use with the present invention.

Following preparation of the lipid composition, a lipid complex is formulated by combining the lipid composition and adenovirus in a pharmaceutically acceptable carrier solution by rapid mixing. Rapid mixing can be done by using a T-mixing apparatus or ethanol injection of the therapeutic agent. In certain embodiments a DOTAP:Cholesterol lipid composition is prepared by the methods described and combined with the adenovirus. The lipid composition is provided in an amount to encapsulate the adenovirus and result in a colloidal suspension of the lipoplex.

A lipid composition may comprise DOTAP and cholesterol, a cholesterol derivative or a cholesterol mixture and adenovirus for delivery into disease cells or into cells near the disease cells. The treatment of a disease, in one embodiment, involves the intravenous administration of adenovirus lipoplex to a patient. The lipoplex treatment of the patient delivers the adenovirus, resulting in the destruction of the neoplastic cells.

The initial lipid mixture will preferably be of powdered lipid components that can be weighed and mixed to appropriate molar ratios. The components can be anionic lipids, cationic lipids, neutral lipids, sterols, and/or other hydrophobic molecules in ratios necessary to produce the desired characteristics of the final lipid composition or complex. The actual composition of the lipid mixture will be determined by the properties required for efficient delivery of the agent(s) to the desired target cells by the desired means of administration, described in detail below. Components of the composition can be mixed to provide various molar ratios. The molar concentrations of any component of the lipid composition can be from about 0.5 mM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, and 50 mM. The molar ratio of any two of the components can be from about 1:100. 1:50, 1:25, 1:20:1:18, 1:16, 1:15, 1:14, 1:13, 1:12, 1:11, 1:10, 1:9, 1:8, 1:7, 1:6, 1:5, 1:4, 1:3, 1:2, 1:1, 1:0.5, 1:0.25, 1:0.1, 1:0.05, or 1:0.01.

The lipid compositions are capable of carrying biologically active agents. The lipid composition can sequester toxic compounds to reduce free concentrations in the serum, protect compounds from degradative agents in the body, and/or mask antigenic components to reduce the immunogenicity of the agent.

A preferred lipid composition is DOTAP and cholesterol or a cholesterol derivative. The ratio of DOTAP to cholesterol, cholesterol derivative or cholesterol mixture may about 4:1 to 1:4, 3:1 to 1:3, more preferably 2:1 to 1:2, or 1:1. The DOTAP or cholesterol concentrations may be between about 1 to 8 mM, 2 to 7 mM, 3 to 6 mM, or 4 to 5 mM. Cholesterol derivatives may be readily substituted for the cholesterol or mixed with the cholesterol in the present invention. A number of cholesterol derivatives are known to the skilled artisan. Cholesterol acetate and cholesterol oleate may be used.

An effective amount of the therapeutic agent is determined based on the intended goal, for example, elimination of tumor cells. The term “unit dose” refers to physically discrete units suitable for use in a subject, each unit containing a predetermined-quantity of the therapeutic composition calculated to produce the desired responses, discussed above, in association with its administration, i.e. the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

Parenteral formulations may be in the form of liquid solutions or suspensions; for oral administration, formulations may be in the form of tablets or capsules; and for intranasal formulations, in the form of powders, nasal drops, or aerosols.

The engineered viruses of the present invention may be administered directly into animals, or alternatively, administered to cells that are subsequently administered to animals.

As used herein, the term in vitro administration refers to manipulations performed on cells removed from an animal, including, but not limited to, cells in culture. The term ex vivo administration refers to cells that have been manipulated in vitro, and are subsequently administered to a living animal. The term in vivo administration includes all manipulations performed on cells within an animal. In certain aspects of the present invention, the compositions may be administered either in vitro, ex vivo, or in vivo.

In vivo administration of the compositions of the present invention are contemplated. An example includes direct injection of tumors with the instant compositions by intracranial administration to selectively kill tumor cells.

C. Combination Therapy

Tumor cell resistance to DNA damaging agents represents a major problem in clinical oncology. One goal of current cancer research is to find ways to improve the efficacy of chemo- and radiotherapy, as well as other conventional cancer therapies. One way is by combining such traditional therapies with oncolytic adenovirus therapy. Traditional therapy to treat retinoblastoma or other cancers may include enucleation (removal of the affected eye), external beam irradiation, episcleral plaques (i.e. 125I plaques), xenon arc and argon laser photocoagulation, cryotherapy, immunotherapy and chemotherapy. The choice of treatment is dependent on multiple factors, such as, 1) multifocal or unifocal disease, 2) site and size of the tumor, 3) diffuse or focal vitreous seeding, 4) age of the patient or 5) histopathologic findings (The Genetic Basis of Human Cancer, 1998).

The herpes simplex-thymidine kinase (HS-tk) gene, when delivered to brain tumors by a retroviral vector system, successfully induced susceptibility to the antiviral agent gancyclovir (Culver et al., 1992). In the context of the present invention, it is contemplated that adenoviral therapy could be used similarly in conjunction with anti-cancer agents, including chemo- or radiotherapeutic intervention. It also may prove effective to combine mutant oncolytic virus therapy with immunotherapy, as described above.

To kill cells, inhibit cell growth, inhibit metastasis, inhibit angiogenesis or otherwise reverse or reduce the malignant phenotype of tumor cells, using the methods and compositions of the present invention, one could contact a “target” cell with a mutant oncolytic virus and optionally at least one other agent. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the mutant oncolytic agent and the other includes the agent.

Oncolytic adenoviral therapy may also be combined with immunosuppression. The immunosuppression may be performed as described in WO 96/12406. Examples of immunosuppressive agents include cyclosporine, FK506, cyclophosphamide, and methotrexate.

Alternatively, the mutant oncolytic virus treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either mutant oncolytic virus or the other agent will be desired. Various combinations may be employed, where mutant oncolytic virus is “A” and the other agent is “B”, as exemplified below:

| | | | | | | | |
|---|---|---|---|---|---|---|---|
| A/B/A | B/A/B | B/B/A | A/A/B | B/A/A | A/B/B | B/B/B/A | B/B/A/B |
| A/A/B/B | A/B/A/B | A/B/B/A | B/B/A/A | B/A/B/A | B/A/A/B | B/B/B/A | |
| A/A/A/B | B/A/A/A | A/B/A/A | A/A/B/A | A/B/B/B | B/A/B/B | B/B/A/B | |

Other combinations are contemplated. Again, to achieve cell killing, both agents are delivered to a cell in a combined amount effective to kill the cell.

Agents or factors suitable for use in a combined therapy are any chemical compound or treatment method with anticancer activity; therefore, the term “anticancer agent” that is used throughout this application refers to an agent with anticancer activity. These compounds or methods include alkylating agents, topoisomerase I inhibitors, topoisomerase II inhibitors, RNA/DNA antimetabolites, DNA antimetabolites, antimitotic agents, as well as DNA damaging agents, which induce DNA damage when applied to a cell.

Examples of chemotherapy drugs and pro-drugs include, CPT11, temozolomide, platin compounds and pro-drugs such as 5-FC. Examples of alkylating agents include, inter alia, chloroambucil, cis-platinum, cyclodisone, flurodopan, methyl CCNU, piperazinedione, teroxirone. Topoisomerase I inhibitors encompass compounds such as camptothecin and camptothecin derivatives, as well as morpholinodoxorubicin. Doxorubicin, pyrazoloacridine, mitoxantrone, and rubidazone are illustrations of topoisomerase II inhibitors. RNA/DNA antimetabolites include L-alanosine, 5-fluoraouracil, aminopterin derivatives, methotrexate, and pyrazofurin; while the DNA antimetabolite group encompasses, for example, ara-C, guanozole, hydroxyurea, thiopurine. Typical antimitotic agents are colchicine, rhizoxin, taxol, and vinblastine sulfate. Other agents and factors include radiation and waves that induce DNA damage such as, γ-irradiation, X-rays, UV-irradiation, microwaves, electronic emissions, and the like. A variety of anti-cancer agents, also described as “chemotherapeutic agents,” function to induce DNA damage, all of which are intended to be of use in the combined treatment methods disclosed herein. Chemotherapeutic agents contemplated to be of use, include, e.g., adriamycin, bleomycin, 5-fluorouracil (5-FU), etoposide (VP-16), camptothecin, actinomycin-D, mitomycin C, cisplatin (CDDP), podophyllotoxin, verapamil, and even hydrogen peroxide. The invention also encompasses the use of a combination of one or more DNA damaging agents, whether radiation-based or actual compounds, such as the use of X-rays with cisplatin or the use of cisplatin with etoposide.

In treating pre-cancer or cancer according to the invention, one would contact the cells of a precancerous lesion or tumor cells with an agent in addition to the mutant oncolytic virus. This may be achieved by irradiating the localized tumor site with radiation such as X-rays, UV-light, γ-rays or even microwaves. Alternatively, the cells may be contacted with the agent by administering to the subject a therapeutically effective amount of a pharmaceutical composition comprising a compound such as, adriamycin, bleomycin, 5-fluorouracil, etoposide, camptothecin, actinomycin-D, mitomycin C, podophyllotoxin, verapamil, or more preferably, cisplatin. The agent may be prepared and used as a combined therapeutic composition, or kit, by combining it with a mutant oncolytic virus.

Agents that directly cross-link nucleic acids, specifically DNA, are envisaged to facilitate DNA damage leading to a synergistic, anti-neoplastic combination with a mutant oncolytic virus. Cisplatinum agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally. Bleomycin and mitomycin C are other anticancer agents that are administered by injection intravenously, subcutaneously, intratumorally or intraperitoneally. A typical dose of bleomycin is 10 mg/m², while such a dose for mitomycin C is 20 mg/m².

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Immunotherapy may be used as part of a combined therapy, in conjunction with mutant oncolytic virus-mediated therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. Antibodies specific for CAR, integrin or other cell surface molecules, may be used to identify cells that the adenovirus could infect well. CAR is an adenovirus receptor protein. The penton base of adenovirus mediates viral attachment to integrin receptors and particle internalization.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that local, regional delivery of mutant oncolytic virus to patients with retinoblastoma-linked cancers, pre-cancers, or hyperproliferative conditions will be a very efficient method for delivering a therapeutically effective gene to counteract the clinical disease. Similarly, the chemo- or radiotherapy may be directed to a particular, affected region of the subjects body. Alternatively, systemic delivery of expression construct and/or the agent may be appropriate in certain circumstances, for example, where extensive metastasis has occurred.

In addition to combining mutant oncolytic virus therapies with chemo- and radiotherapies, it also is contemplated that combination with other gene therapies will be advantageous. For example, targeting of the mutant oncolytic virus in combination with the targeting of p53 at the same time may produce an improved anti-cancer treatment. Any tumor-related gene conceivably can be targeted in this manner, for example, p21, Rb, APC, DCC, NF-1, NF-2, BCRA2, p16, FHIT, WT-1, MEN-I, MEN-II, BRCA1, VHL, FCC, MCC, ras, myc, neu, raf, erb, src, fms, jun, trk, ret, gsp, hst, bcl and abl.

It is further contemplated that the therapies described above may be implemented in combination with all types of surgery. Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection, systemic administration, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well. Furthermore, in treatments involving more than a single treatment type (i.e., construct, anticancer agent or surgery), the time between such treatment types may be about 1, 2, 3, 4, 5, 6, 7, 8, 9, 10, 11, 12, 13, 14, 15, 16, 17, 18, 19, 20, 21, 22, 23, or about 24 hours apart; about 1, 2, 3, 4, 5, 6, or 7 days apart; about 1, 2, 3, 4, or 5 weeks apart; and about 1, 2, 3, 4, 5, 6, 7, 8, 9, 10, 11, or 12 months apart, or more.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

It also should be pointed out that any of the foregoing therapies may prove useful by themselves. In this regard, reference to chemotherapeutics and non-mutant oncolytic virus therapy in combination also should be read as a contemplation that these approaches may be employed separately.

IV. Screening for Anti-Tumor Activity in Adenoviral Constructs Using Animal Models

Animal models may be used as a screen for tumor suppressive effects of oncolytic adenovirus of the invention. Preferably, orthotopic animal models will be used so as to closely mimic the particular disease type being studied and to provide the most relevant results. One type of orthotopic model involves the development of an animal model for the analysis of microscopic residual cancer cell(s) and microscopic seeding of body cavities.

The first step in the development of an exemplary animal model is to create a tissue flap in the experimental animal. The term “tissue flap” means any incision in the flesh of the animal that exposes the target tissue. It is generally preferred that an incision be made in the dorsal flank of an animal, as this represents a readily accessible site. However, it will be understood that an incision could well be made at other points on the animal, and the choice of tissue sites may be dependent upon various factors such as the particular type of therapeutics that are being investigated.

Once a target tissue site is exposed, cancer cells, either individually or in tumors, are contacted with the tissue site. Cancer cell application may be achieved simply using any convenient applicator. Naturally, this procedure will be conducted under sterile conditions.

In a particular example, 1×10⁷ cells are inoculated into the exposed tissue flap of a nude mouse. Those of skill in the art will be able to readily determine, for a given purpose, what the appropriate number of cells will be. The number of cells will be dependent upon various factors, such as the size of the animal, the site of incision, the replicative capacity of the tumor cells themselves, the time intended for tumor growth, the potential anti-tumor therapeutic to be tested, and the like. Although establishing an optimal model system for any particular type of tumor may require a certain adjustment in the number of cells administered, this in no way represents an undue amount of experimentation. For example, this can be accomplished by conducting preliminary studies in which differing numbers of cells are delivered to the animal and the cell growth is monitored following resealing of the tissue flap. Naturally, administering larger numbers of cells will result in a larger population of residual tumor cells. Those skilled in the area of animal testing will appreciate that such optimization is required.

Other orthotopic animal models are well known in the art. The skilled artisan will readily be able to adapt or modify each particular model for his intended purpose without undue experimentation.

V. Screening for a Defective Rb Pathway

With adenovirus delta24 and other mutant adenovirus that are unable to bind Rb, it is necessary for the Rb pathway to be defective in order for the cell to transcribe and translate viral proteins. The Rb pathway is required to be defective in the sense that it is not able to repress the transcription-activating activity of E2F. E2F activates the transcription of cellular genes and adenoviral DNA if its activity is not repressed. Examples of ways in which E2F could escape repression include, but are not limited to, Rb not being able to bind E2F (i.e. E1A binding to Rb), overexpression of E2F, less Rb than E2F and situations in which Rb remains phosphorylated.

A. Antibodies

Antibodies can be used to detect adenoviral proteins (e.g., E1A), Rb, and other proteins of the Rb pathway. In certain aspects of the invention, one or more antibodies may be produced that are immunoreactive with multiple antigens. These antibodies may be used in various diagnostic or therapeutic applications, described herein below.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)2, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Monoclonal antibodies (MAbs) are recognized to have certain advantages (e.g., reproducibility and large-scale production). The invention thus provides for monoclonal antibodies of the human, murine, monkey, rat, hamster, rabbit and even chicken origin. Due to the ease of preparation and ready availability of reagents, murine monoclonal antibodies may be preferred.

However, “humanized” antibodies are also contemplated, as are chimeric antibodies from mouse, rat, or other species, bearing human constant and/or variable region domains, bispecific antibodies, recombinant and engineered antibodies and fragments thereof.

The methods for generating monoclonal antibodies (MAbs) and polyclonal antibodies are well known in the art. Briefly, a polyclonal antibody is prepared by immunizing an animal with a composition in accordance with the present invention and collecting antisera from that immunized animal. MAbs may be readily prepared through use of well-known techniques, such as those exemplified in U.S. Pat. No. 4,196,265, incorporated herein by reference. Typically, this technique involves immunizing a suitable animal with a selected immunogen composition (e.g., a purified or partially purified protein, polypeptide, or peptide) be it a wild-type or mutant composition. The immunizing composition is administered in a manner effective to stimulate antibody producing cells. Following immunization, somatic cells with the potential for producing antibodies, specifically B lymphocytes (B cells), are selected for use in the MAb generating protocol. The antibody-producing B lymphocytes from the immunized animal are then fused with cells of an immortal myeloma cell, generally one of the same species as the animal that was immunized. This culturing provides a population of hybridomas from which specific hybridomas are selected. The selected hybridomas would then be serially diluted and cloned into individual antibody-producing cell lines, which clones can then be propagated indefinitely to provide MAbs. It is also contemplated that a molecular cloning approach may be used to generate monoclonals. Alternatively, monoclonal antibody fragments encompassed by the present invention can be synthesized using an automated peptide synthesizer, or by expression of full-length gene or of gene fragments in E. coli.

1. Antibody Conjugates

Certain embodiments of the invention provide antibodies to antigens and translated proteins, polypeptides and peptides that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”. Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ^{59.}iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

B. Immunodetection Methods

Adenoviral gene expression in a population of cells will be determined by western blot analysis using polyclonal rabbit antibodies as probes to adenoviral proteins. The level of viral proteins detected would indicate whether viral protein expression is occurring in the cell. Immunodetection using monoclonal antibodies that recognize various epitopes within the Rb protein or a protein of the Rb pathway can be used to see if Rb or a protein in the Rb pathway has been mutated.

In still further embodiments, the present invention concerns immunodetection methods for binding, purifying, removing, quantifying and/or otherwise generally detecting biological components such as protein(s), polypeptide(s) or peptide(s) involved in adenoviral replication or the cellular Rb or p53 pathways. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle MH and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing a protein, polypeptide and/or peptide of interest, and contacting the sample with a first antibody in accordance with the present invention under conditions effective to allow the formation of immunocomplexes. The immunobinding methods also include methods for detecting and quantifying the amount of an antigen component in a sample and the detection and quantification of any immune complexes formed during the binding process.

In terms of antigen detection, the biological sample analyzed may be any sample that is suspected of containing an antigen, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any ORF antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

Another known method of immunodetection takes advantage of the immuno-PCR (Polymerase Chain Reaction), the method of which is well known in the art.

1. ELISAs

Immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, an antibody that recognizes an antigen is immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that recognizes the antigen and is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes.

Detecting the bound immune complexes includes quantifying the amount of label, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated (e.g., using a spectrophotometer).

2. Immunohistochemistry

Antibodies may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990, all of which are incorporated herein by reference).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

C. Nucleic Acid Detection

In addition to their use in directing the expression of adenoviral or Rb pathway proteins, polypeptides and/or peptides, the nucleic acid sequences disclosed herein have a variety of other uses. For example, they have utility as probes or primers for embodiments involving nucleic acid hybridization. They can be used to determine whether viral genes are being transcribed. In certain embodiments of the invention adenoviral genes may be transcribed in cells with a mutant Rb or p53 pathways. Nucleic acid detection may be used to determine if there is a mutation within the Rb gene, p53 gene or other genes encoding proteins of the Rb pathway. The DNA sequences of genes of the present invention may be determined by methods known in the art to identify mutations within the sequence.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to adenoviral or Rb pathway proteins are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCRTM) which is described in detail in U.S. Pat. Nos. 4,683,195, 4,683,202 and 4,800,159, and in Innis et al., 1990, each of which is incorporated herein by reference in their entirety.

A reverse transcriptase PCR™ amplification procedure may be performed to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application 320308, incorporated herein by reference in its entirety. U.S. Pat. No. 4,883,750 describes a method similar to LCR for binding probe pairs to a target sequence. A method based on PCRTM and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application 2 202 328, and in PCT Application PCT/US89/01025, each of which is incorporated herein by reference in its entirety.

Qbeta Replicase, described in PCT Application PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

Other nucleic acid amplification procedures include Strand Displacement Amplification (SDA), disclosed in U.S. Pat. No. 5,916,779; transcription-based amplification systems (TAS), including nucleic acid sequence based amplification (NASBA) and 3SR (Kwoh et al., 1989; Gingeras et al., PCT Application WO 88/10315, incorporated herein by reference in their entirety). Davey et al., European Application 329 822 disclose a nucleic acid amplification process involving cyclically synthesizing single-stranded RNA (“ssRNA”), ssDNA, and double-stranded DNA (dsDNA), which may be used in accordance with the present invention.

Miller et al., PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. Other amplification methods include “race” and “one-sided PCR” (Frohman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. See Sambrook et al., 1989. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Other Assays

Other methods for screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCRTM (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

D. Biopsy

A tumor may be biopsied and the above tests performed upon it to determine whether the cells have a functional Rb pathway. An example of a biopsy protocol is as follows. The stereotactic biopsy is the precise introduction of a metal probe into the brain tumor, cutting a small piece of the brain tumor, and removing it so that it can be examined under the microscope. The patient is transported to the MRI or CAT scan suite, and the frame is attached to the scalp under local anesthesia. The “pins” of the frame attach to the outer table of the skull for rigid fixation (frame will not and can not move from that point forward until completion of the biopsy). The scan (MRI or CT) is obtained. The neurosurgeon examines the scan and determines the safest trajectory or path to the target. This means avoiding critical structures. The spatial co-ordinates of the target are determined, and the optimal path is elected. The biopsy is carried out under general anesthesia. A small incision is created over the entry point, and a small hole is drilled through the skull. The “dura” is perforated, and the biopsy probe is introduced slowly to the target. The biopsy specimen is withdrawn and placed in preservative fluid for examination under the microscope. Often the pathologist is present in the biopsy suite so that a rapid determination of the success of the biopsy can be made.

E. Diagnostic and In Vitro Uses

Any of the methods above can be used in the present invention for diagnostic and in vitro uses. The oncolytic adenoviruses of the present invention may be used in diagnostic assays to detect the presence of cells with a defective Rb and/or p53 pathway. A sample of cells could be infected with the oncolytic adenovirus of the present invention and after an incubation period, the number of cells exhibiting adenovirus replication can be quantified to determine the number of neoplastic cells in the sample. This may be useful to determine if the adenovirus would be effective in treating the tumor from a patient from which a cell sample was taken. Other uses are to diagnose a neoplasm as having a defective Rb and/or p53 pathway and to evaluate tumor cell load following treatment.

Alternate diagnostic uses and variations include an adenovirus with a Rb binding mutation in the E1A or an E1B55 kD-mutation and a reporter gene to score whether cells have been transformed by detecting reporter gene expression. Expression of the reporter gene can be correlated with a phenotype of adenoviral replication indicating a lack of a functional Rb and/or p53 pathway.

VI. Polynucleotide and Polypeptide Variants

A. Polypeptide Variants

Amino acid sequence variants of the polypeptides discussed above and throughout this application, specifically including E1A and E1B55 kD, may be substitutional, insertional or deletion variants. Deletion variants lack one or more residues of the native protein and may result in reduction or elimination of protein expression. In the present invention, a polypeptide variant may be a deletion of a Rb binding region. For example, the deletion of all or part of the CR2 region of the E1A adenoviral protein or other regions of proteins, including the deletion of entire proteins, that enable adenovirus to circumvent p53 and/or Rb pathways. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of a particular polypeptide, such as E1A, provided the biological activity of the protein is maintained.

The term “functionally equivalent codon” is used herein to refer to codons that encode the same amino acid, such as the six codons for arginine or serine, and also refers to codons that encode biologically equivalent amino acids (see Table 1, below).

TABLE 1
CODON TABLE

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that amino acid and nucleic acid sequences may include additional residues or nucleotides, such as additional N- or C-terminal amino acids or 5′ or 3′ nucleic acid sequences. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

In making amino acid changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

a) Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

2. Polynucleotides

The present invention concerns polynucleotides that are capable of expressing a protein, polypeptide, or peptide discussed above, such as one derived from the E1A gene product or a heterologous gene. Mutations to E1A may be at any location within the E1A gene that disrupts Rb/E1A interaction. The discussion below focuses on E1A but it is understood that the discussion may be applied to other adenoviral polynucleotides of the present invention.

A DNA segment encoding an E1A polypeptide refers to a DNA segment that contains wild-type, polymorphic, or mutant E1A polypeptide-coding sequences. Included within the term “DNA segment” are a polynucleotide or polynucleotides, DNA segments smaller than a polynucleotide, and recombinant vectors. Recombinant vectors may include plasmids, cosmids, phage, viruses, and the like. In certain embodiments recombinant adenoviruses are contemplated. In particular, an adenovirus with an altered E1A and/or E1B region, in particular a full or partial deletion of the E1B55 kD protein encoding nucleic acid sequence of adenovirus.

Mutations of the invention may be point mutants in the nucleic acid or they may be mutations affecting or be affecting at least or at most 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1560, 1565, 1570, 1575, 1585, 1590, 1595, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000 or more contiguous nucleotides of nucleic acid disclosed herein, such as SEQ ID NO:1 or SEQ ID NO:3. The mutation may be a substitution, insertion, or deletion. In some embodiments, a mutation introduces a stop codon or introduces a frame shift that results in a premature stop codon. It is further contemplated that nonfunctional polypeptides may be encoded by polynucleotides, such as truncated polypeptides. Moreover, it is contemplated the polynucleotides of the invention may be mutated to produce a polypeptide that lacks 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 or more contiguous amino acids, including the full-length polypeptide, including the polypeptides of SEQ ID NO:2 and SEQ ID NO:4.

As used in this application, the term “polynucleotide” refers to a nucleic acid molecule of greater than 3 nucleotides. Therefore, a “polynucleotide encoding an E1A polypeptide” refers to a DNA segment that contains a wild-type, a polymorphic, or a mutant E1A polypeptide; similarly, a “polynucleotide encoding wild-type E1A” refers to a DNA segment that contains wild-type E1A polypeptide coding nucleic acid or DNA sequences.

Similarly, a polynucleotide comprising an isolated or purified wild-type, polymorphic, or mutant E1A gene refers to a DNA segment including wild-type, polymorphic, or mutant E1A polypeptide coding sequences and, in certain aspects, regulatory sequences, isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid encoding E1A may contain a contiguous nucleic acid E1A sequence encoding all or a portion of E1A of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, or more nucleotides, nucleosides, or base pairs.

“Isolated substantially away from other coding sequences” means that the gene of interest, for example the polynucleotide encoding a wild-type, a polymorphic, or a mutant E1A polypeptide, forms part of the coding region of the DNA segment, and that the DNA segment does not contain large portions of naturally-occurring coding DNA. Of course, this refers to the DNA segment as originally isolated, and does not exclude genes or coding regions later added to the segment by human manipulation.

The nucleic acid segments used in the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode full-length E1A or encode a truncated or mutant version of E1A. The transcript may then be translated into a protein. Alternatively, a nucleic acid sequence may encode a full-length E1A protein sequence with additional heterologous coding sequences, for example to allow for purification of E1A, transport, secretion, or post-translational modification of E1A.

In a non-limiting example, one or more nucleic acid or adenoviral constructs may be prepared that include a contiguous stretch of nucleotides identical to or complementary to the E1A gene. A nucleic acid construct may comprise 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 750, 800, 850, 900, 950, 1,000, to 1,550 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values). Non-limiting examples of intermediate lengths include 11, 12, 13, 16, 17, 18, 19 and 1,001, 1002. Non-limiting examples of intermediate ranges include 3 to 32 and 150 to 1,550.

The DNA segments used in the present invention may encompass biologically functional equivalent E1A proteins and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to decrease the antigenicity of the protein or to inhibit binding to a given protein.

a) Cloning, Gene Transfer, and Expression

Adenoviruses of the present invention can be constructed using methods known in the art and described herein. Expression requires that appropriate signals be provided which include various regulatory elements, such as enhancers/promoters that may be derived from both viral and mammalian sources that drive host cell expression of the genes of interest. Elements designed to optimize messenger RNA stability and translatability in host cells also are defined.

3. Regulatory Elements

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Promoters that permit expression of a protein of interest generally under most conditions and in most cell types is termed constitutive, and an example of this is the CMV promoter. A tissue-specific promoter is a regulatable promoter that is allows expression only in particular tissues or cells. Tables 2 and 3 list several elements/promoters that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

TABLE 2
ENHANCER/PROMOTER

Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
γ-Globin
β-Globin
c-fos
c-HA-ras
Insulin
Neural Cell Adhesion Molecule (NCAM)
α1-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 3

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X |
| | poly(rc) |
| Adenovirus 5 E2 | E1A |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | PMA |
| Thyroid Stimulating Hormone α | Thyroid Hormone |
| Gene | |
| Insulin E Box | Glucose |

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

4. Selectable Markers

The markers listed below can be inserted as a heterologous sequence in the adenovirus genome. In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also may be employed. The selectable marker employed is not believed to be important, so long as it is capable of being expressed simultaneously with the nucleic acid encoding a gene product. Further examples of selectable markers are well known to one of skill in the art.

5. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message. An example of such a construct is described in U.S. Pat. No. 5,665,567, which is herein incorporated by reference.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single vector and a single selectable marker.

### VII. EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

A. In Vitro Site-Directed Mutagenesis.

The adenovirus Δ24 was constructed from a modified adenovirus type 5 genome as follows. The shuttle plasmid pXC 1, which contains the adenovirus 5 sequences from 22 to 5790 nucleotides, was modified by using phosphorylated mutagenic primers to loop out and delete 24 bases, from 923 to 946, of the adenovirus type 5 corresponding to amino acid residue sequence of LTCHEAGF of the E1A protein.

B. Confirmation of the Δ24-E1A by Sequencing the Genome of the Adenovirus.

A 1-kb fragment of the E1A region of the adenoviral construct was amplified by polymerase chain reaction (PCR) using sense (5′-GATTGGCCACCATGAGACATA TTATCTGC-3′) (SEQ ID NO: 5) and antisense (5′-CTGTGGCCATTTAACACGCCATGCA-3′) (SEQ ID NO: 6) primers. Subsequently, 1 μg of concentrated PCR sample was added to 1 μl of each primer (primer dilution 1:3 of 0.1 μg/μl) and the volume was brought to 9.5 μl with distilled water. This mixture of PCR fragments and primers was added to a fluorescein-labeled reaction mix and sequenced in a 373A automated DNA sequencer.

C. Construction of the Δ24 by Homologous Recombination in 293 Cells.

To generate the mutant adenovirus, the shuttle vectors pXC 1-A24, which carries the mutant E1A region of the adenovirus, and pBHG10, which contains the rest of the adenoviral genome except for the E3 region, were cotransfected by liposome-mediated method into 293 cells to allow homologous recombination. To generate infectious virions, individual plaques were isolated and amplified, and the viral titers were determined by plaque assays.

D. Adenoviruses.

Other vectors used included the UV-D24 adenovirus, a D24 virus inactivated by ultraviolet light; Ad5CMV-pA, a E1-deleted adenovirus that carries no exogenous genes (Alemany et al., 1996); Ad5CMV-Rb, a E1-deleted adenovirus that carries the Rb cDNA (Fueyo et al., 1998c); Ad5CMV-GFP, a vector that can express the green fluorescent protein (Alemany et al., 1997); Ad5CMV-p21, that carries the p21 cDNA (Gomez-Manzano et al., 1997); dl1520, a replication competent adenovirus with a partial deletion of the E1B gene (Barker and Berk, 1987).

E. Cell Lines and Infection Conditions.

The human glioma cell lines U-251 MG (homozygous deletion of p16 locus, Arap et al., 1995), U-373 MG (lack of expression of p16 protein, Fueyo et al., 1998b), and U-87 MG (homozygous deletion of p16 locus, Arap et al., 1995); the human sarcoma cell line Saos-2 (lack of wild-type Rb expression, Huang et al., 1998); and the normal lung fibroblast line CCD32-Lu were obtained from the American Type Culture Collection. The D-54 MG human glioma cells (lack of expression of p16 protein, Fueyo et al., 1996a) were a generous gift from Dr. Bigner (Duke University, Durham, N.C.). The cell lines were infected as described previously (Gomez-Manzano et al., 1996; Fueyo et al., 1998b, which are incorporated herein by reference).

F. Detection of the E1A Protein by Western Blotting.

Twenty four hours after infection, total protein from U-251 MG cells infected with Ad5CMV-pA, dl1520, or Δ24 at a dose of 100 MOI, were analyzed with anti-E1A using the following method. Total cell lysates were prepared by incubating cells for 1 h at 4° C. in RIPA buffer (150 mM NaCl, 1% Triton X-100, 1% sodium deoxycholate, 0.1% sodium dodecyl sulfate (SDS), 20 mM EDTA, and 50 mM Tris, pH 7.4) at different times after the infection. Thirty micrograms of protein from each sample was then subjected to 7% SDS-Tris-glycine gel electrophoresis. The membrane was blocked with 3% nonfat milk, 0.05% Tween 20, 0.9% NaCl, and 50 mM Tris, pH 7.5, and incubated with the following primary antibodies: rabbit anti-adenovirus-2 E1A protein 13 S-5, and mouse anti-human actin IgG. The secondary antibodies were horseradish peroxidase-conjugated donkey anti-rabbit IgG and horseradish peroxidase-conjugated donkey anti-mouse IgG. The membranes were developed according to Amersham's electro-chemiluminiscence protocol.

G. Immunoprecipitation of E1A/Host Protein Complexes.

Total lysates from U-251 MG cells infected with Δ24, dl1520, or Ad5CMV-pA at a dose of 100 multiplicity of infection (MOI) were immunoprecipitated using an antibody agarose-conjugated anti-E1A. Ten milligrams of antibody agarose conjugated rabbit anti-adenovirus-2 E1A protein 13 S-5, was added to 500 mg of total cell proteins from cells infected with Δ24, dl1520, or Ad5CMV-pA, and incubated at 4° C. overnight. The pellet was collected by centrifugation, washed with RIPA, and subjected to 7% SDS polyacrylamide gel electrophoresis followed by immunoblotting with goat anti-human Rb C-15 antibody (Fueyo et al., 1998c).

H. Cell Viability Assay.

Cells were seeded at 10⁵ cells per well in six-well plates and allowed to grow for 20 h. Cells were infected with Δ24, Ad5CMV-pA, Ad5CMV-Rb, or UV-Δ24 at the indicated MOI. After 7-14 days of incubation the cell monolayers were fixed with methanol and stained with 0.2% crystal violet. The experiment was performed 3 times for each cell line. Trypan blue experiments were performed as described elsewhere (Fueyo et al., 1998b, incorporated herein by reference).

I. Transfer of Exogenous Wild-Type Rb or p21.

Characterization of the Rb- and p21-adenovirus and its infection into cells was performed as described elsewhere (Gomez-Manzano et al., 1997; Fueyo et al., 1998c, both of which are incorporated herein by reference).

J. Infectivity Experiments in Normal Cells.

CCD32-Lu lung fibroblasts were infected with 10-100 MOI of the Ad5CMV-GFP; and 3 days later, the cells were examined in terms of both interference and fluorescent contrast. The percentage of infected cells was determined by counting the number of green fluorescent cells among 500 examined cells.

K. Viral Replication Experiments.

Viral production was quantified by plaque assay. Six days after infection, cells were scraped into culture medium and lysed by 3 cycles of freezing and thawing. Cell lysates were clarified by centrifugation and the supernatant was serially diluted in medium for the infection of 293 cells in 6-well dishes. After 1 h of incubation at 37° C., the infected cells were overlaid with 3 ml of 1.25% SeaPlaque agarose (FMC, Rockland, Me.) in DMEM/F12 (10% fetal bovine serum). Additional agarose was added to each dish 4 days later. Plaques were visualized at 7 days after infection.

L. Electron Microscopy.

Four days after infection, pellets from U-251 MG cells infected with UV-Δ24, or Δ24 were fixed in 2% glutaraldehyde and post-fixed for 1 h in 1% osmium tetroxide. Samples were counter-stained with uranyl acetate and lead citrate and examined under a transmission electron microscope (Gomez-Manzano et al., 1996, incorporated herein by reference)

M. Animal Studies.

Mice were acclimated and caged in groups of 5 or less. All mice were fed animal chow and water ad libitum. Animals were anesthetized with methoxyflurane before all procedures and were observed until fully recovered from the effect of the anesthesia. Pieces of approximately 3 mm in diameter of D-54 MG pre-established tumors, or 10⁷ D-54MG parental cells were inoculated subcutaneously into the right flanks of 6- to 8-week-old athymic BALB/c-nu/nu (nude) mice. When tumors reached at least 5 mm in diameter, they were injected with either Δ24 or UV-Δ24 at one of the indicated doses. The largest (a) and smallest (b) diameters of each tumor were measured, and tumor volume was calculated using the formula (a)×(b²)×(0.4) (Attia and Weiss, 1996). When the tumors had reached the maximum acceptable size, the mice were euthanized, and their tumors were excised and analyzed by histological fixation and staining with hematoxylin and eosin. Each experiment was performed separately on at least 5 different animals from each group. Tumor responses to each treatment were compared by the 2-tailed t-student test.

### Example 2

Intracranial Administration

A sterotactic headframe is implanted under local anesthesia. After intravenous gadolinium administration, a stereotactic MRI is performed for localization of the tumor mass. Histological analysis is performed on the specimen obtained from a stereotactic biopsy to confirm the presence of a tumor. The specimen is also analyzed by immunohistochemistry for the Rb protein and RT-PCR to analyze the nucleotide sequence of the Rb gene.

The injection of the Δ24 composition is made using a silastic catheter. One ml of liquid containing 3×10⁸ to 1×10¹¹ viral particles of Δ24 is injected over 10 minutes. The needle is flushed with saline to assure delivery of the virus (the volume of the catheter is determined prior to needle placement to assure that only the 1 ml of virus and not the saline is delivered). After injection, the catheter is cut at the level of the skull and closed with a hemoclip. A non-contact CT is performed to verify the site of injection and identify any acute asymptomatic hematomas.

Δ24 is also injected into the residual tumor cavity following tumor resection. Δ24 is distributed throughout the tumor wall by the following method. A grid of approximately 1 cm squares is established using suture material. Each 1 cm² square is injected with Δ24 using a 20 g blunt tip Dandy needle attached to a 1 cc syringe. The needle is inserted 1 to 2 cm into the parenchyma and Δ24 is infused over 1 minute per injection. Minimal irrigation is used after injection.

### Example 3

Intracranial Administration Using an Implanted Guide-Screw System.

Δ24 was administered intracranially to mice using an implanted guide-screw system by the method of Lang (Lang et al., 2000). The system comprises a 2.6 mm guide screw with a central 0.5 mm diameter hole that accepts the 26 gauge needle of a Hamilton syringe. The skin was prepared with iodine and a 2 to 3 mm incision was made to the right of midline and anterior to the interauroal line. A small hand-controlled twist drill (1 mm in diameter) was used to make a 1 mm in diameter hole that is 2.5 mm lateral and 1 mm anterior to the bregma. The screw was implanted into the drill hole. The sterilized guide screw was rotated into the hole until it was flush with the skull using a specially devised screwdriver (Plastics One) that holds the guide screw. The shaft of the screw protrudes through the dura and into the brain surface. A cross shaped stylet was used to cap the screw between treatments. Injection of Δ24 was done freehanded using a Hamilton syringe and a 26 gauge needle fitted with a cuff that was cemented to the lower end of the needle so that only 2 mm of the needle tip protrudes from the distal end of the screw when the sleeve is flush with the top of the screw. The tip of the needle is 3.5 mm below the skull surface. Δ24 was injected using a Hamilton syringe placed through the hole in the guide screw.

### Example 4

The E1A-mutant Δ24 adenovirus produces anti-cancer effect in vitro

The replication-competent Δ24 virus is a human adenovirus 5 that contains a 24-bp deletion in its E1A region (FIG. 2). This deletion did not produce a stop codon, and the Δ24 could express a mutant E1A protein. Immunoprecipitation experiments confirmed however that the mutant E1A protein could not form complexes with the Rb protein (Whyte et al., 1988; Whyte et al., 1989).

The infectivity of the adenovirus has been previously tested in a variety of glioma and sarcoma cell lines that have disruptions in the p16/Rb pathway (Fueyo et al., 1996a; Fueyo et al., 1998c). The Δ24 adenovirus had similar effects on the viability of U-251 MG, D-54 MG, U-87 MG, U-373 glioma cells and Saos-2 osteosarcoma cells. Thus, treating any of these cells with the mutant adenovirus at a multiplicity of the infection (MOI) of 10 produced complete monolayer cytolysis (85% or more) within 14 days for most of the glioma cell lines and cytopathic effect within 7 days for the Saos-2 osteosarcoma line. These results were confirmed by both Trypan blue and crystal violet viability assays. In the crystal violet assays, plated glioma cells were infected with Δ24 adenovirus at indicated MOI. The plates were stained with crystal violet 7 days (U-251 MG), 9 days (D-54 MG), or 14 days (U-87 MG) after infection. Dose-dependence experiments revealed that 5 MOIs of the Δ24 adenovirus were enough to induce a noticeable cytopathic effect, although the timing and intensity of the oncolytic effect varied slightly among the cell lines tested (FIG. 3). For example, by 7 days after treatment with 5 MOI of Δ24 adenovirus the oncolytic effect was complete in U-251 MG. In contrast, D-54 MG cells required 10 MOI to obtain a complete monolayer lysis, and U-87 MG cells required 5 MOI and 14 days of infection.

### Example 5

The Replication of Δ24 is Responsible for the Anti-Cancer Effect

Since wild-type adenoviruses can infect and lyse quiescent fibroblasts (Bischof et al., 1996; Heise et al., 1997; Rothman et al., 1998; Hay et al., 1999), it was determined whether the Δ24 had lost its ability to replicate in and kill normal cells. For these experiments, CCD32-Lu lung fibroblasts were arrested in G₁ phase after being seeded at a low density of 5×10³ cells per well in six-well plates, cultured with medium plus 1% serum, and infected with 10 plaque-forming units (PFU) per cell (the maximum dose used to treat cancer cells) of Δ24 or UV-Δ24 (control) adenovirus. Eight days after the infection no differences were found in the numbers of Δ24- and control-infected cells (3.3×10³±0.11; 2.5×10³+0.07, respectively). Moreover, Δ24 infection did not result in changes in the morphology of fibroblasts. These results did not reflect an inability of the adenovirus to infect normal cells because these lung fibroblasts could be infected with 50 MOI (>70% of the cells) of Ad5CMV-GFP. Next, U-251 MG and U-373 MG cells plated at 5×10³ cells per well in six-well plates were infected and cultured with medium plus 1% serum, with 10 PFU/cell of Δ24 or UV-Δ24 adenovirus. Seven days after the infection, a massive cytopathic effect was observed, with a 94.5±4.6% and 90.1±4.1% decrease in viability of Δ24-infected-U-251 MG and U-373 MG glioma cells, respectively, with respect to the UV-Δ24-infected cells. To confirm that the Δ24 adenovirus has different effects on the viability of normal and cancer cells, replication assays in which 293 cells were incubated with cell lysates of either normal or cancer cells were performed. To obtain the cell lysates from either CCD32-Lu, D-54 MG or U-251 MG, cells were plated at the same density (10⁴ cells per well in six-well plate), infected with 10 MOIs of either the Δ24 or the UV-Δ24 adenovirus, and allowed to grow for 6 days. After the infection with D-54 MG or U-251 MG cell lysates, the 293 cells showed destruction of the monolayer cultures in as soon as 2 days. Four days after infection, numerous viral particles were observed in the cancer cells. In contrast, 293 cell did not die when the lysates were from normal cells. These results suggested that the Δ24 produced progeny virus more efficiently in cancer cells than in normal cells.

Plaque assays were used to quantify the cytopathic effect in terms of viral titers. Virus yields were determined by titer in 293 cells at 6 days after the infection of D-54 MG, U-251 MG and CCD32-Lu with 1 MOI of either the Δ24 adenovirus or the UV-Δ24 control adenovirus. The Δ24 titer was 60 and 80 times higher than the initial dose in 2 independent experiments with the D-54 MG lysates and 200 and 400 times higher in the U-251 MG lysate experiments. By contrast, the initial titer did not increase when the lysates were from CCD32-Lu cells (FIG. 4). The UV-Δ24 infected cells did not induce production of new virus. The differences in the viral titers observed in the experiments with D-54 MG and U-251 MG agreed with the differences in cytopathic effect observed in the crystal violet assays. These experiments showed that the Δ24 adenovirus replicated efficiently in glioma cells and that normal fibroblasts restricted the spread of the Δ24.

### Example 6

Transfer of Rb Restricts the Δ24-adenovirus Mediated Cytolysis

The ability of the mutant adenovirus to replicate in cells arrested and expressing wild-type Rb was evaluated. For these experiments the osteosarcoma cell line Saos-2 was used. Saos-2 has a well-characterized disruption of the Rb pathway (Huang et al., 1988) can be easily infected with adenovirus (Fueyo et al., 1998c), and has a well-characterized response to the transfer of an exogenous Rb (Huang et al., 1988; Fueyo et al., 1998c). Saos 2 cells were infected with 100 MOI of an adenoviral vector carrying the exogenous wild-type Rb cDNA, or the Ad5CMV-pA adenovirus, and 72 hours later were infected with 10 MOIs of either the Δ24 or the UV-Δ24 adenovirus. Cells that had been pretreated with a vector control, Ad5CMV-pA, were sensitive to the lytic effect of the Δ24 adenovirus with a complete cytopathic effect (90.5±2.6% decrease in viability) observed within 5 days after the viral infection. By contrast, cells infected with the Ad5CMV-Rb acquired an oncolytic-resistant phenotype with an 88.7±3.1% increase in viability, that persisted for at least for 10 days after infection with the Δ24 adenovirus (FIG. 5).

Since, the effect of Δ24 is theoretically restricted by cell-cycle factors, the changes in the cell-cycle profile of Saos-2 cells were monitored in parallel with the experiments described above. Saos-2 cells infected with the Ad5CMV-Rb were arrested in G₁ phase by the third day after infection (77.2±3% of Rb-infected cells versus 59.7±3% of control-infected cells). The accumulation of cells in G₁ correlated with the observed growth inhibition (71.9±3.2%) relative to cells infected with an adenovirus control. Flow-cytometric analyses of the cell cycle performed on the fifth day after Δ24 infection showed that the number of cells in S phase was 2.4- and 5.5-fold lower in the Rb-pretreated cells, in comparison with adenovirus control-pretreated cells, in 2 independent experiments. These results show that restoration of Rb rendered Δ24 unable to efficiently induce entry of cells into S phase and therefore precluded viral replication.

The ability of the cyclin-dependent kinase inhibitor p21, a regulator of Rb function, to reduce the effect of the Δ24 on the viability of wild-type Rb cells was examined to confirm the virus-suppressive effect of the Rb protein. D-54 MG cells were infected with 100 MOI of an adenoviral vector carrying the exogenous wild-type p21 cDNA or Ad5CMV-pA, and 3 days later were infected with the Δ24 virus at 10 MOI. For cells that had not been infected with Ad5CMV-p21 vector, a total cytopathic effect (74.2±2.9% decrease of viability) was observed 5 days after the infection with Δ24. In contrast, p21-pretreatment provided partial protection against the Δ24 adenovirus as reflected by a 64.2±4% increase in viability. As was true for the Rb-transfer experiment, growth inhibition (40±1.9%) was apparent 3 days after infection with Ad5CMV-p21. Thus, p21-treated cells showed an overrepresentation in the G₁ phase of the cell cycle in comparison with cells treated with the adenovirus control (91.4±0.4% and 75.3±0.9%, respectively).

### Example 7

Treatment with Δ24 Induces Entry of Cells into S Phase and Cell Death

Flow cytometric analysis (performed as described in Gomez-Manzano et al., 1996; Fueyo et al., 1998c) of the DNA content of the Δ24-infected cells showed an accumulation of cells in the S phase by the third day after the infection. Thus, 81±10.1% of Δ24-infected U-251 MG cells were in the S phase of the cell cycle versus 10±2.3% of the UV-Δ24-infected cells. Similar results were observed in D-54 MG cells, and 77.4% and 77.3% of Δ24-infected cells were in S phase (in comparison with 14.5% and 15.3% of the control-infected cells) in 2 independent experiments. Accumulation of cells in the S phase was also observed in Δ24-infected Saos-2 cells. At 7 days after infection, cytopathic effect was widespread. The majority of the cells detached from the dish and cellular debris were numerous. However, there was only a small population of cells in the sub-G₁ area as assessed by flow cytometric analyses. Thus, 7.72±3.8% of Δ24-infected U-251 MG were in the sub-G₁ region (versus 1.7±0.1 of control infected cells). The discrepancy between the morphological and flow-cytometric data suggested that the virus-mediated cell death was probably due to both cell lysis and apoptosis. Electron microscopy showed necrosis and apoptosis features in the infected cells. Δ24 adenovirus induced cell death even in mutant-p53 cells such U-251 MG glioma cells (Gomez-Manzano et al., 1996) and Saos-2 (Masuda et al., 1987). These results indicated that although p53 can cooperate with other viral and cell proteins to enhance adenovirally-mediated cell death (Ridgway et al., 1997), wild-type p53 status was not a major determinant of efficient Δ24 replication and thus its oncolytic effect.

### Example 8

The Δ24 Adenovirus Suppresses Tumor Growth In Vivo

To study the anti-tumor effect of Δ24 in vivo, the growth of D-54 MG tumors treated with Δ24 was compared with the growth of tumors those treated with an UV-inactivated Δ24 control virus in a nude mice model. Tumor size was measured periodically until tumors in the control animals achieved the maximum acceptable burden. In the first group of experiments, pre-established D-54 MG xenografts were implanted subcutaneously in nude mice, and when tumors reached at least 5 mm in diameter, tumors were treated with either Δ24 (n=6) or UV-Δ24 (n=6) at a dose of 5×10⁸ PFU (plaque forming units) per animal every 5 days for a total of 5 times. All of the Δ24-treated tumors showed an 83.8% growth inhibition (P<0.01, t-student, double sided), with tumor regression in 2 of the 6 mice tested. Tumors showing regression have been followed for over two months without evidence of regrowth. Hematoxylin-eosin staining revealed that, after Δ24 treatment, the remaining tumor mass consisted largely in necrotic tissue. Similar results were obtained in tumors formed after the subcutaneous injection of 10⁷ D54 MG cells in the right flank of nude mice. These experiments involved the intratumoral injection of 10⁹ PFU of either Δ24 (n=5) or UV-Δ24 (n=5) every 5-7 days for 4 doses. With these doses, an 85.7% reduction in the size of treated tumors relative to the size of the control tumors (P<0.05, t-student, double sided) were observed. As true for the previous group, 2 of the 5 Δ24-treated mice showed tumor regression.

In addition to the multiple injection experiments, the effect of a single low dose of the Δ24 adenovirus on tumor growth was investigated. Tumors in 6 mice were injected with 10⁷ PFU of the adenovirus control, and tumors in 5 mice were treated with a similar dose of Δ24. At the end of the experiment, tumors in the Δ24-treated group were 66.3% smaller than those in the UV-Δ24 treated animals (P<0.005, t-student, double sided). Despite the low viral dose, all of the Δ24-treated tumors displayed growth inhibition (FIGS. 6A., 6B. and 6C.). None of the mice used in these experiments exhibited any sign of systemic toxicity including weight loss, abdominal swelling, hunched posture, or diarrhea nor did they die before the end of the experiment.

### Example 9

In Vivo Treatment of U-251 MG Glioma with Δ24 Adenovirus

Malignant gliomas represent an excellent mode for adenoviral therapy, as adenovirus may be injected directly into these relatively localized tumors. To test the anti-tumor properties of Δ24 adenovirus in vivo in a relevant animal model, established U-251 MG gliomas were stereotactically injected with Δ24 or an UV-inactivated Δ24. In these experiments, 6 immunodeficient nude mice were treated with a single intratumoral injection of 10⁸ pfu of Δ24 and other 6 were treated with a similar dose of UV-Δ24. The treatment with Δ24 improved survival significantly (p<0.05). Similar results were obtained in two independently performed experiments.

U-251 MG cells express high levels of CAR and RGD-related integrins, two receptors that are required for adenovirus and anchorage and internalization. For that reason, the anti-glioma effect of Δ24 and Δ24 RGD in U-87 MG tumors was tested. These tumors express a low amount of CAR, but a high level of integrins (FIG. 7). U-87 MG cells are one of the most resistant cell lines to adenovirus infection and adenovirus replication. In fact, treatment of U-87 MG cells with 10 pfu/cell (a dose that induces cytopathic effect in less than 8 days in other human glioma cell lines, such as U-251 MG) require a minimum of 14 days to induce a cytopathic effect greater than 80% of the extension of the culture.

Forty nine animals bearing intracranially implanted U-87 MG tumors (5×10⁵ cells) were treated with three intratumoral injections (days, 3, 5 and 8 of the experiment; day 1, implantation of the tumor) of 1.5×10⁸ pfu of Δ24, Δ24-RGD, UV-Δ24, ONYX-015 (E1B deleted), or PBS (no virus). All the animals in the PBS, ONYX-015, and UV-Δ24 died by day 25 of the experiment. However, animals treated with either Δ24 or Δ24-RGD survived longer. These experiments were performed again with similar results. Together, these experiments show that 38.7% of the Δ24 or Δ24-RGD-treated animals did not present any symptom of the disease and were sacrificed 140 days (first experiment) and 170 days (second experiment) after the implantation of the tumor.

The brain of any animal that died because of the tumor or that was sacrificed was removed, embedded in paraffin and prepared for microscopic examination. All of the control animals that died before day 25 had big-volume tumors that were responsible for the animal's death. The tumors of the Δ24 or Δ24-RGD animals that survived more than 25 days but died because of the tumor showed a mass with three different areas. A central area of necrosis surrounded by a second zone of infected cells (cells showed viral inclusions), and a peripheral zone of uninfected or low infected tumor. This pattern of three, more or less concentric areas, suggested that the adenovirus induced lyses of cells and tumor necrosis in the area of injection and spread form this area to the periphery of the tumor. In some cases, some viral particles arrived to the limit between the tumor and the normal mouse tissue. Immunohistochemistry analyses of the infected tumors were performed to confirm replication of the virus. Using anti-hexon antibodies it was shown that cells with viral inclusions observed by light microscopy were positive for viral proteins. Since the production of hexon proteins is a late event in the adenoviral cell cycle, the detection of these adenoviral proteins in the cells indicated viral replication. To confirm the ability of the adenovirus to replicate within the U-87 MG glioma tumors, an immunohistochemistry analysis using anti-E1A antibodies was performed. Expression of the E1A protein is required for adenovirus replication. These experiments showed that the infected cells were positive for exogenous E1A adenoviral protein. These analyses strongly confirmed the presence of a replication competent adenovirus in the treated tumors. The immunohistochemistry analyses with either anti-hexon or anti-E1A antibodies was unable to detect viral particles in the brain cells of the mice, which do not allow adenoviral replication. The examination of the brain of the animals sacrificed between the days 140 and 170 showed no presence of tumors. Instead, there were changes compatible with tumor cure, in the site of the tumor injection. These findings included dystrophic calcification and mycrocystic formation. Immunohistochemical analyses of these regions did not detect viral particles.

The animal model used in the U-87 MG experiments showed that all untreated animals died by day 25. The in vivo conditions for viral spread are more astringent than in vitro. In contrast with an in vitro, homogenous collection of cells, many of them growing in monolayer, an in vivo tumor constitute an spherical system encompassing a mixture of human and mouse cells, mainly of endothelial origin. These mouse cells are unable to host adenoviral replication and may constitute a physical barrier for adenoviral spread. In addition, within human glioblastoma there are areas of necrosis that may also limit cell-to-cell spread of the adenovirus. Although severely immunodeficient, the in vivo model probably allows a minimum anti-viral response by the tumor cells and by the immune system of the mouse. Consistent with these observations another replication competent adenovirus (ONYX-015) is unable to replicate and kill U-87 MG tumors. These observations suggest that the Δ24 and Δ24-RGD adenoviruses would be unable to improve animal survival in mice bearing U-87 MG tumors. Using the U-87 MG model, neither radiotherapy, nor treatment of tumors with adenovirus carrying powerful anti-tumor molecules such as E2F-1 or p53, were unable to improve survival with the same efficiency than Δ24 and Δ24-RGD. The complete elimination of the tumor in 38.7% of the animals is an unexpected result, because in previous studies using a subcutaneous animal model (a system in which the treatment can be more accurately injected into the tumor), multiple injections of Δ24 (five doses of 5×10⁸) to treat D-54 MG (a human glioma cell line that, contrarily to U-87 MG, can be easily infected by adenovirus) subcutaneous tumors produced complete suppression of growth in a lower percentage of cases.

### Example 10

Enhancement of Δ24 Anti-Cancer Effect Using Chemotherapy.

The characterization of a conditionally replicative adenovirus, Delta24, with anti-cancer effect in human gliomas is described. Since infection of human glioma cells with Delta24 results in accumulation of cells in the S phase of the cell cycle, studies of the anti-cancer effect of Delta24 in combination with CPT-11 and Temozolomide (TMZ), which are both S-phase specific chemotherapeutic agents.

Human glioma cells were infected with 10 MOI of Delta24 adenovirus and two-three days later cells were collected and examined for DNA content by flow cytometry. The accumulation of cells in the S phase of the cell cycle was striking in comparison with the control cells infected with an UV-inactivated Delta24.

### Example 11

Cell-Cycle Profiles of Cells Treated with Delta24 and CPT-11 or TMZ.

The effect of the combination of Delta24 adenovirus and CPT-11 or TMZ on cell cycle progression was analyzed by comparison of the cell cycle profiles of U-251 MG cells treated with Delta24 or UV-inactivated Delta24 after treatment with no drug, with CPT-11, or TMZ. Each drug was administered at a concentration corresponding to the IC₅₀ dose in U-251 MG cells. Representative cell cycle profiles 48 h post-treatment are shown in FIG. 8. In the absence of active Delta24, treatment with CPT-11 resulted in an accumulation of cells in G2/M phase. This arrest was completely abrogated in cells pre-treated with Delta24, where most of the cells were in S phase. Treatment of U-251-MG exposed to an UV-inactivated Delta24 with TMZ also resulted in a dramatic G2/M-phase arrest. However, as with CPT-11, pre-treatment with Delta24 of U-251-MG caused complete abrogation of the TMZ-induced G2/M-phase arrests (FIG. 8).

### Example 12

Sequential Administration of Delta24 and CPT-11 or TMZ Enhances the Chemotherapeutic Effect.

In view of the fact that CPT-11 and TMZ are predominately S-phase specific drugs, it was hypothesized that agents that promote S phase entry and accumulation would enforce CPT-11 or TMZ effects. This issue is of clinical importance because cell cycle stimulators are entering clinical trials in combination with chemotherapy.

In these experiments, a sequential schedule was designed based on the hypothetical mechanism of the Delta24 mediated potentiation of the drug effect (i.e.: induction of S phase) and previous data that indicate a dramatic accumulation of cells into the S phase after 48 h of Delta24 infection. Thus, cells were treated with Delta24 adenovirus or UV-inactivated Delta24, and 48 hours later, treated with the chemotherapeutic agents in a dose-dependent experiment. Cell viability was assessed by MTT assay. CPT-11-mediated anticancer effect was more prominent in cells that had been pretreated with Delta24, as shown in FIGS. 9, 10, and 11. Similar results were obtain with the combination Delta24 and TMZ (FIGS. 12, 13, and 14).

### Example 13

Synergistic Effect of Delta24 and CPT-11 or TMZ.

To determine the synergistic effect of Delta24 with conventional chemotherapy, a sensitivity study was carried out on these glioma cells. Cytotoxicity was tested using cell viability assay assessed by MTT of the Delta24 (compared to UV-inactivated Delta24), with or without CPT-11 or TMZ at concentrations lower than the IC₅₀. Synergistic effect was observed with the combination of the oncolytic and chemotherapeutic agents (FIGS. 15 and 16).

Taken together our data indicate that infection of human glioma cells with Delta-24 adenovirus induces cell-cycle mediated chemosensitivity to both CPT-11 and temozolomide. In addition, sequential administration of Delta-24 adenovirus and CPT-11 or temozolomide was shown to result in synergistic anti-cancer effect.

### Example 14

CB001 Double-Mutant Conditionally Replicating Adenovirus

The double mutant adenovirus CB001 comprises partial deletions of the E1A and E1B adenoviral genes (FIG. 17). The E1A deletion comprises amino acids 122 to 129 (as in the Delta24 construct), affecting the CR2 domain, and renders the mutant E1A protein unable to bind Rb. In addition to the E1A modification, the CB001 contains a deletion in the E1B region (2426 to 3328 bp. from the adenoviral genome; Gene Bank Access number: NC_{—}001406, incorporated herein by reference) that prevents expression of the wild type E1B55K protein and prevents repression of p53 tumor suppressor pathway. The E1B19K gene, whose protein product suppresses apoptosis, is not affected by this deletion.

### Example 15

CB001 Anti-Cancer Effect In Vitro

The CB001 mutant virus was tested for its ability to induce cytopathic effect in human malignant glioma cell lines (D-54 MG, U-373 MG, U-251 MG). A quantitative assay, the MTT assay, was used for this analysis. Dose dependence experiments showed that the cytopathic effect is highly reproducible and dose dependent in the three cell lines tested (FIG. 18). Furthermore, in another set of experiments performed independently of the MTT assay, CB001-induced cytopathic effect (CPE) was monitored by staining the remaining cells on the plate at the end of the experiment with crystal violet. These experiments confirmed that CB001 exhibited increasing cytopathic effect with increasing MOIs in the cell lines tested (FIG. 19). In addition to the MTT and crystal violet, daily monitoring of the infected cultured by light microscopy showed a time related effect. Thus, while inactivated-adenovirus-infected cells showed no cytopathic effect, CB001-infected primary cells showed a weak cytopathic effect at 3 days postinfection and a strong cytopathic effect at 8 to 9 days postinfection as evidenced by the detachment of the monolayer and the formation of round, light-refractile bodies.

### Example 16

Toxicity in Arrested Normal Human Astrocytes.

In these experiments, replication assays were performed on normal cells infected with CB001, Delta24, RA55 (an adenovirus carrying only the E1B deletion present in CB001) or wild type adenovirus (Ad300). To test the ability of CB001 to acquire a replication phenotype in quiescent normal human astrocytes, these cultures were grown as monolayers and arrested by serum starvation. Cells were then infected with CB001, Delta24, RAS5, Ad300 or UV-inactivated-CB001 (control) adenovirus in a dose-dependent experiment. CB001 infection did not change the morphology of astrocytes as assessed by optic microscopy, and did not affect their viability ten days after the infection (FIG. 20). To quantify the viral replication in quiescent normal cells compared to tumor cells 293 were used cells to determine viral titer three days after the infection of Normal Human Astrocytes with 1 MOI of each adenovirus. The Ad300 titer was 10.75 higher than the initial dose (media from three independent experiments). By contrast, the titer did not increase when cells were infected with CB001. The UV-inactivated Delta24 infected cells did not induce production of new virus (FIG. 21). Therefore, compared to tumor cells, CB001 did not appear to replicate in non-proliferating normal cells.

### Example 17

Toxicity in Proliferating Human Astrocytes.

The relevance of the double-mutant was established in experiments using actively dividing astrocytes. In contrast to Ad300, CB001 showed distinct patterns of replication in the proliferating astrocytes. Ad300 and CB001 were tested in proliferating normal human astrocytes at a dose of 1 MOI. Seventy-two hours after infection, viral replication assay was performed to analyze the replication profile of both constructs. In two independent experiments, CB001 was unable to efficiently replicate in these cultures. The ratio Ad300 replication to CB001 replication was 390 and 4200 in these two experiments. Together both toxicity experiments and anti-cancer effects demonstrate an attractive therapeutic index of the CB001 construct for therapeutic use in gliomas.

### Example 18

Anti-Cancer Effect In Vivo.

The therapeutic efficacy of CB001 was examined in vivo by growing D54 MG human glioblastoma multiforme cells as tumor xenografts in athymic mice. The tumor cells were injected intracranially into the right basal ganglia of each mouse, and three days later, after establishment of microscopically evident tumors, the tumors were directly injected used a screw-guided surgical model with a single dose of 1.5×10⁸ pfu/tumor of CsCl-purified CB001, RA55, Delta24 or ultraviolet (UV)-inactivated Ad300 virus, as a negative control. After treatment, animal were observed for signs of toxicity and survival. Treatment of D-54 MG tumors with CB001 resulted in significant improvement of animal survival as compared with UV-inactivated Ad300 (p<0.01) (FIG. 22). The results obtained demonstrate that a single dose of CB001 administered intracranially improves the survival of glioma-bearing nude mice.

To ensure that the improvement of survival was due to CB001 virus replication, sections were analyzed from the excised tumors for Ad5 capsid proteins. When animals died, the brains were removed, fixed in 4% formaldehyde and sectioned. Histological examination of brains from control animals demonstrated that all died from intracranial tumor growth. The immunohistochemical staining of the tumors remaining in animals treated with CB001 with antibodies to Ad5 hexon protein indicates that CB001 replicated and disseminated throughout the D-54 MG tumors.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-61. (canceled)

62. A method for treating a brain tumor in a patient comprising:
a. identifying a patient having a brain tumor comprising one or more cells having a defective Rb pathway; and
b. contacting the tumor with an oncolytic adenovirus that encodes an E1A polypeptide that cannot bind Rb.

63. The method of claim 62, wherein contacting is by intracranial administration.

64. The method of claim 63, wherein intracranial administration is by perfusion.

65. The method of claim 63, wherein intracranial administration is by injection.

66. The method of claim 62, wherein the composition is directly injected into the tumor.

67. The method of claim 62, wherein the E1A polypeptide comprises a deletion of one or more amino acids, an insertion of one or more amino acids, or substitution of one or more amino acids.

68. The method of claim 67, wherein the E1A polypeptide comprises a deletion of one or more amino acids.

69. The method of claim 68, wherein amino acids 122-129 of SEQ ID NO:2 are deleted.

70. The method of claim 62, wherein a cell of the tumor is a metastasis.

71. The method of claim 70, wherein the metastasis is derived from a lung, breast, ovary, cervix, pancreas, stomach, colon, skin, larynx, bladder, or kidney cancer.

72. The method of claim 62, wherein the tumor is a neurocytoma, neutroblastoma, primitive neuroectodermic tumor (PNET), medulloblastoma, glioma, sarcoma, astrocytoma, oligodendroglioma, anaplastic glioma, ependymonas, meningiomas, pineal region tumor, choroid plexus tumor, or neuroepithelial tumor.

73. The method of claim 72, wherein the tumor is a glioma.

74. The method of claim 62, wherein the administration occurs more than once.

75. The method of claim 74, wherein the composition is administered at least three times to the patient.

76. The method of claim 62, wherein the adenovirus encodes a fiber protein comprising a RGD motif.

77. The method of claim 62, wherein the adenovirus encodes a heterologous polypeptide.

78. The method of claim 77, wherein the heterologous polypeptide is a reporter polypeptide.

79. The method of claim 77, wherein the heterologous polypeptide is a therapeutic polypeptide.

80. The method of claim 79, wherein the heterologous polypeptide is an enzyme to convert a pro-drug into an active chemotherapy drug.

81. The method of claim 62, further comprising administering to the patient a second therapy, wherein the second therapy is chemotherapy, immunotherapy, surgery, radiotherapy, immunosuppresive agents, or gene therapy with a therapeutic polynucleotide.

82. The method of claim 81, wherein the second therapy is administered to the patient before, during, after or any combination thereof, administration of the composition comprising the oncolytic adenovirus.

83. The method of claim 81, wherein the chemotherapy comprises an alkylating agent, mitotic inhibitor, antibiotic, or antimetabolite.

84. The method of claim 81, wherein the chemotherapy comprises CPT-11, temozolomide, or a platin compound.

85. The method of claim 81, wherein radiotherapy comprises X-ray irradiation, UV-irradiation, y-irradiation, or microwaves.

86. The method of claim 62, wherein from about 10³ to about 10¹⁵ viral particles are administered to the patient.

87. The method of claim 86, wherein from about 10⁵ to about 10¹² viral particles are administered to the patient.

88. The method of claim 86, wherein from about 10⁷ to about 10¹⁰ viral particles are administered to the patient.


SEQUENCE LISTING (6 sequences)

SEQ ID NO: 1 - DNA, 561 bp, Adenovirus
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtga

SEQ ID NO: 2 - protein, 186 aa, Adenovirus
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSK

SEQ ID NO: 3 - DNA, 1270 bp, Large T-Antigen
  CDS (1)..(1266)
atgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgag

SEQ ID NO: 4 - protein, 422 aa, Large T-Antigen
MNVVQVAELYPELRRILTITEDGQGLKGVKRERGACEATEEARNLAFSLMTRHRPECITFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 5 - DNA, 29 bp, Artificial Sequence
  Description of Artificial Sequence Synthetic Primer
gattggccaccatgagacatattatctgc

SEQ ID NO: 6 - DNA, 25 bp, Artificial Sequence
  Description of Artificial Sequence Synthetic Primer
ctgtggccatttaacacgccatgca